(19) United States
(12) Patent Application Publication
(10) Pub. No.: US 2010/0311757 A1
(43) Pub. Date: Dec. 9, 2010

(54) Salarins And Tulearins, Compositions And Uses Thereof

(75) Inventors: Yoel Kashman, Tel Aviv-Yafo (IL); Maurice Aknin, St.-Denis (FR); Amira Rudi, Ramat Hasharon (IL); Ashgan Bishara, Tira (IL); Drorit Neumann, Ramat Hasharon (IL); Nathalie Ben Califa, Herzelia (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignees: RAMOT AT TEL-AVIV UNIVERSITY LTD., Tel Aviv (IL); Université De La Réunion, Saint Denis De La Rèunion (FR)

(21) Appl. No.: 12/680,405

(22) PCT Filed: Sep. 25, 2008
(86) PCT No.: PCT/IL08/01293

§ 371 Date: Mar. 26, 2010

Related U.S. Application Data
(60) Provisional application No. 61/123,366, filed on Apr. 8, 2008.

(30) Foreign Application Priority Data
Sep. 26, 2007 (GB) 0718825.3

(51) Int. Cl. A61K 31/395; C07D 498/04; C07D 313/00; A61K 31/424; C07D 487/04; A61K 31/5025; A61P 35/00

(52) U.S. Cl. 514/248; 540/455; 540/456; 549/271; 514/450; 514/375; 544/236

(57) ABSTRACT

Salarines and Tulearins isolated from Fascaplysinopsis sp. sponge and synthetic derivatives thereof are provided.

### FIELD OF THE INVENTION

This invention is concerned with the isolation of nitrogenous macrolides, compositions comprising them and their use as medicaments.

### BACKGROUND

Many organisms, especially soft corals, sponges and tunicates, provide many secondary metabolites and exhibit a varying degree of biological activity [1]. An important family of these metabolites is the macrolide family. Two members of the macrolide family are Madangolide [2] and Laingolide A [3], which have been isolated from the cyanobacterium Lyngbya bouillonii and their structures have been studied and established.

The macrolide family includes members which are used as drugs, typically antibiotics [4,5]. The activity of the various macrolides is believed to stem from the macrolide ring to which one or more deoxy sugars, usually cladinose and desosamine, are attached.

### REFERENCES

[1] Faulkner, D. Nat. Prod. Rep. 1997, 14, 259-302 and references therein.
[2] Klein, D.; Braekman, J. C.; Daloze, D.; Hoffmann, L.; Castillo, G.; Demoulin, V. J. Nat. Prod. 1999, 62, 934-936.
[3] Klein, D.; Braekman, J. C.; Daloze, D.; Hoffmann, L.; Castillo, G.; Demoulin, V. Tetrahedon Lett. 1996, 37, 7519-7520.
[4] Keicho, N.; Kudoh, S. Am J Respir Med. 2002, 1, 119-131.
[5] Lopez-Boado, Y. S.; Rubin, B. K.; lung Curr Opin Pharmacol. 2008, 8, 286-2910.

### BRIEF DESCRIPTION OF THE FIGURES

FIG. 1 shows COSY (—) and key correlation of HMBC ( [symbol] ) of Salarin A.

FIG. 2 shows COSY (—) and key correlation of HMBC ( [symbol] ) of Salarin B.

FIG. 3 shows COSY (—), key correlation of HMBC ( [symbol] ) of Tulearin A.

FIGS. 4A to 4C provide selective NOE correlations for Salarin A (FIG. 4A), Salarin B (FIG. 4B) and Tulearin A (FIG. 4C).

FIGS. 5A and 5B present the effect of Salarin A on UT7 (FIG. 5A) and Ba/F3 (FIG. 5B) cell lines.

FIGS. 6A and 6B present the effect of Tulearin A on K562 (FIG. 6A) and UT7 (FIG. 6B) cell lines.

FIG. 7 shows the ¹H-NMR spectrum of Salarin A (500 MHz, Acetone-d₆).

FIG. 8 shows ¹³C-NMR spectrum of Salarin A (100 MHz, Acetone-d₆).

FIG. 9 shows the COSY spectrum of Salarin A (500 MHz, Acetone-d₆).

FIG. 10 shows the HMBC spectrum of Salarin A (500 MHz, Acetone-d₆).

FIG. 11 shows the ¹H-NMR spectrum of Salarin B (500 MHz, C₆D₆).

FIG. 12 shows the ¹³C-NMR spectrum of Salarin B (100 MHz, C₆D₆).

FIG. 13 shows the COSY spectrum of Salarin B (500 MHz, C₆D₆).

FIG. 14 shows the HMBC spectrum of Salarin B (500 MHz, C₆D₆).

FIG. 15 shows the ¹H-NMR spectrum of Tulearin A (500 MHz, Acetone-d₆).

FIG. 16 shows the ¹³C-NMR spectrum of Tulearin A (100 MHz, Acetone-d₆).

FIG. 17 shows the COSY spectrum of Tulearin A (500 MHz, Acetone-d₆).

FIG. 18 shows the HMBC spectrum of Tulearin A (500 MHz, Acetone-d₆).

FIG. 19 shows the ¹H-NMR spectrum of Salarin C (400 MHz, C₆D₆).

FIG. 20 shows the ¹³C-NMR spectrum of Salarin C (100 MHz, C₆D₆).

FIG. 21 shows schematic proposed biogenesis of Salarin C a. H₂NOH b. Beckmann rearrangement. c. Enolization together with the rearrangement and closure of the oxazole.

FIG. 22 shows schematic possible conversion of Salarin C to Salarins A and B.

FIGS. 23 A and 23B show COSY (—), key HMBC correlations ( [symbol] ) and ¹⁵N-HMBC ( [symbol] ) (FIG. 23A) and (b) Selective NOESY ( [symbol] )(FIG. 23B) of Salarin C.

FIGS. 24A and 24B demonstrate the effect of compounds of the invention on cell proliferation. The human erythroleukemic cells lines UT7 (white) and K562 (hatched) and the murine Ba/F3 cell line (black) were incubated with A) compounds at a concentration of 0.5 μg/ml (0.1 μM) for 72 h (FIG. 24A) or with B) at the indicated concentrations of Salarin C for 24 hours (FIG. 24B). Cell viability was measured by the MTT colorimetric assay. Results are presented as % growth of control cells cultured in the absence of Salarin C. Graph represents the mean results±SEM of three identical experiments.

FIG. 25 shows the effect of Salarin C on cell morphology: Ba/F3, K562 and UT7 cell lines were incubated in the absence of Salarin C (control row) or with the indicated concentrations of Salarin C for 24 hours. Cells were viewed in phase contrast microscopy. The size bar indicates a size of 15 um.

FIG. 26 shows the structure of modified Salarins according to the invention.

FIG. 27 shows the structure of modified Tulearins according to the invention.

FIG. 28 shows an exemplary synthetic route for the production of exemplary Salarins according to the invention. For the sake of clarity, only certain sections of the molecules are presented.

FIG. 29 shows an exemplary synthetic route for the production of exemplary Tulearins according to the invention. For the sake of clarity, only certain sections of the molecules are presented.

### SUMMARY OF THE INVENTION

The present invention relates to compounds isolated from the Fascaplysinopsis sp. sponge and to synthetic derivatives thereof. As the present disclosure provides, these compounds have been shown to inhibit proliferation of human and mouse cell lines, providing basis for their use in treating diseases associated with hyperproliferation, e.g., cancer. Thus, the present invention relates to the compounds per se, whether isolated as disclosed or modified based on the parent isolated compounds, as well as to pharmaceutical compositions comprising them, and uses thereof in medicine.

The compounds of the invention have been obtained by isolation from their natural source and their structure was elucidated on the basis of mass spectroscopy (MS) and nuclear magnetic resonance (NMR) analyses, as will be further elaborated on hereinbelow.

Thus, the present invention provides in one of its aspects a compound of the general Formula I, including isomers, e.g., stereoisomers, geometrical isomers; solvates; and salts thereof, whether pharmaceutically acceptable salts or otherwise:

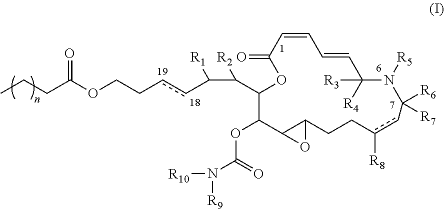

wherein:

R₁ and R₂ each independently is selected from null, —H, —OR₁₁, and —NR₁₂R₁₃,

or

R₁ and R₂ together with the carbon atoms to which they are bonded (i.e., C₁₆ and C₁₇, respectively) form a heterocyclic ring system having 3 or 5 atoms, said heterocyclic ring comprising at least one heteroatom selected from O and N;

R₃ and R₄ each independently is selected from null, —H, —OR₁₄, and —C(O)C₁-C₆-alkyl;

or

R₃ and R₄ together with the carbon atom to which they are bonded (i.e., C₆) form a group selected from a carbonyl group (i.e., C₆═O) and C₆═CR₁₅R₁₆;

R₅ is selected from null, —H and —C(O)C₁-C₆-alkyl;

R₆ and R₇ each independently is selected from null, or together with the carbon to which they are bonded (i.e., C₇) form a carbonyl group (i.e., C₇═O);

where R₃ and R₄ together form C₆═CR₁₅R₁₆ and where R₅ and one of R₆ and R₇ are absent, the N atom bonded to C₇ forms a double bond with C₇ and the other of R₆ and R₇ and one of R₁₅ and R₁₆, together with the carbon atoms to which they are bonded, form a 5-membered heterocyclic ring, said heterocyclic ring comprising one or more atoms selected from N and O;

R₈ is selected from —H and C₁-C₆-alkyl;

R₉ is selected from —H, C₁-C₆-alkyl, C₁-C₆-alkylene-C₆-C₁₀-aryl and C₆-C₁₀-arylene-C₁-C₆-alkyl;

R₁₀ is selected from —H, C₁-C₆-alkyl and —C(O)R₁₇;

R₁₁ is selected from —H and C₁-C₆-alkyl;

R₁₂ and R₁₃ independently of each other is selected from —H and C₁-C₆-alkyl;

R₁₄ is selected from —H and C₁-C₆-alkyl;

R₁₅ and R₁₆ each independently is selected from —H and C₁-C₆-alkyl;

R₁₇ is a C₁-C₆-alkyl;

n is an integer from 0 to 12 (i.e., n being 0, 1, 2, 3, 4, 5, 6, 7, 8, 9, 10, 11 or 12);

the C₈-C₉ bond is a single or double bond (cis or trans);

and

wherein the C₁₈-C₁₉ bond is a single or double bond (cis or trans).

In some embodiments, R₆ and R₇ together with the carbon atom to which they are bonded, i.e., carbon number 7 of the compound of Formula I, designated C₇, form a carbonyl group (C₇=0).

In other embodiments, R₁ and R₂ together with the carbon atoms to which they are bonded form a 3- or 5-membered heterocyclic ring system. The heterocyclic ring system includes at least 2 carbon atoms being those to which R₁ and R₂ are bonded and at least one heteroatom selected from O and N.

The 3- or 5-membered heterocyclic ring system is selected from:

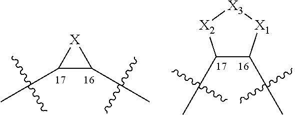

wherein X is selected from —O—, —NH, and —N—C₁-C₆-alkyl; X₁ and X₂ each independently is selected from —O—, —NH, —N—C₁-C₆-alkyl, CH₂, CHhal, C(hal)₂, CH(C₁-C₆-alkyl), C(C₁-C₆-alkyl)₂, CH(C₆-C₁₀-aryl) and C(C₆-C₁₀-aryl)₂; and X₃ is selected from CH₂, CHhal, C(hal)₂, CH(C₁-C₆-alkyl), C(C₁-C₆-alkyl)₂, CH(C₆-C₁₀-aryl) and C(C₆-C₁₀-aryl)₂. hal=halide (i.e., I, Br, Cl, F).

In some embodiments, the heterocyclic ring system is selected from 3- or 5-membered ring systems having at least one oxygen atom, such as a 3-membered epoxide ring, having two carbon atoms (the carbon atoms to which R₁ and R₂ are each bonded) and one oxygen atom (X═O) being bonded to each of these carbon atoms; and a 5-membered ring having 1 or 2 oxygen atoms (one or two of Xᵢ, X₂ and X₃ are O), such as a furyl, and dioxolanyl, said 5-membered ring may optionally be substituted by at least one group selected from C₁-C₆-alkyl, C₆-C₁₀-aryl and a halide. In some embodiments, the 5-membered ring is dioxolanyl. In other embodiments, the 5-membered ring is dimethyldioxolanyl.

In some embodiments, the 3- or 5-membered ring system is an epoxide.

In embodiments where the heterocyclic ring system is an epoxide ring (X═O), the compound of the invention is of the general Formula I-A:

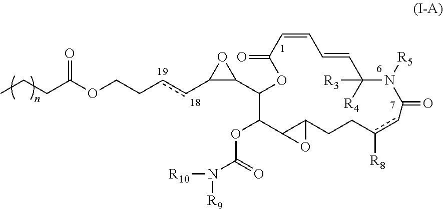

wherein R₃, R₄, R₅, R₈, R₉, R₁₀, and n are as defined hereinabove.

In some embodiments of a compound of general Formula I-A, the C₈-C₉ bond and the C₁₈-C₁₉ bond are each a double bond, i.e., C₈═C₉ and C₁₈═C₁₉, respectively, wherein “C₈”, “C₉”, “C₁₈” and “C₁₉” are designations used herein for carbons 8, 9, 18 and 19, respectively.

In some embodiments, the compound of the invention is a compound wherein the C₈-C₉ bond and the C₁₈-C₁₉ bond are each a double bond, being in the cis or trans configuration. In some embodiments, the C₈-C₉ bond is trans with respect of the ring and the C₁₈-C₁₉ bond is cis. In other embodiments, the C₈-C₉ bond is cis with respect of the ring and the C₁₈-C₁₉ bond is trans. In still other embodiments, both the C₈-C₉ bond and the C₁₈-C₁₉ are cis or both are trans.

In further embodiments, R₃ and R₄ together with the carbon atom to which they are bonded, C₆, form a group selected from a carbonyl group (C₆═O) and C₆═CR₁₅R₁₆. Where R₃ and R₄ together with the carbon atom to which they are bonded form a carbonyl group, the compound of the invention is of general Formula I-B:

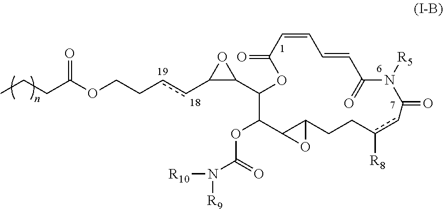

wherein R₅, R₈, R₉, R₁₀ and n are as defined hereinabove.

In some further embodiments, R₅ in Formula I-B is —C(O)C₁-C₆-alkyl and R₉ is —H.

As used herein, “—C(O)C₁-C₆-alkyl” refers to a carbonyl group (the —C(O) segment thereof) being bonded to the N atom situated between C₆ and C₇ and to a C₁-C₆-alkyl.

The term “alkyl” refers to an aliphatic carbon chain, being optionally substituted along the chain, having repeating sp³ hybridized carbon atoms. The chain may be linear or branched. The term “C₁-C₆-alkyl” refers to an alkyl, as defined, having 1, 2, 3, 4, 5 or 6 carbon atoms. Non-limiting examples of such alkyls are methyl, ethyl, propyl, iso-propyl, butyl, iso-butyl, sec-butyl tert-butyl, heptyl, isohexyl and hexyl.

In some embodiments, said alkyl is selected from methyl, ethyl and propyl. In further embodiments, said alkyl is methyl and R₅ is thus —C(O)CH₃.

In further embodiments, R₁₀ is selected from C₁-C₆-alkyl and —C(O)R₁₇,

wherein R₁₇ is a C₁-C₆-alkyl, as defined. In some embodiments, R₁₀ is —C(O)R₁₇ and

R₁₇ is selected from methyl, ethyl and propyl.

The compound of the invention, being a compound of Formulae I, I-A and I-B, is a compound herein designated Salarin A:

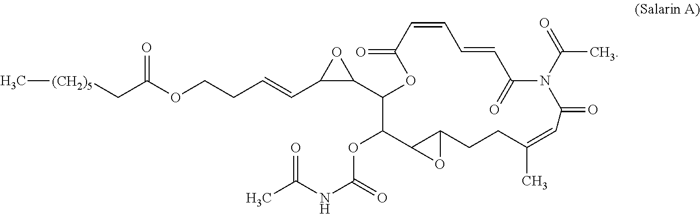

The invention further provides a compound according to Formula I-B, wherein R₅ is —C(O)C₁-C₆-alkyl and R₉ is C₁-C₆-alkyl. In some embodiments, the C₁-C₆-alkyl of R₅ and R₉ are the same, e.g., each being of the same number of carbon atoms, for example R₅ is —C(O)CH₃ and R₉ is —CH₃. In other embodiments, the alkyl of R₅ is different from the alkyl of R₉. For example, R₅ is —C(O)CH₃ and R₉ is an ethyl group.

A compound according to the invention wherein the alkyl of R₅ and that of R₉ are the same is a compound of Formulae I, I-A and I-B, herein designated Salarin A-2:

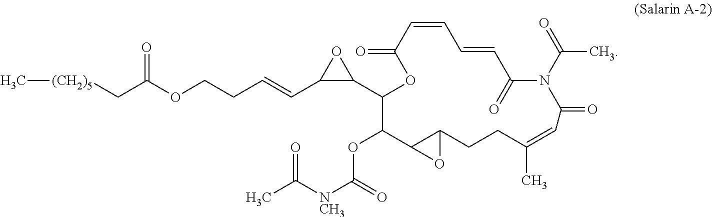

In further embodiments, in a compound of general Formula I-B, R₅ and R₉ may each be a hydrogen atom (—H). One such compound of the present invention is a compound of general Formulae I, I-A and I-B, herein designated Salarin E:

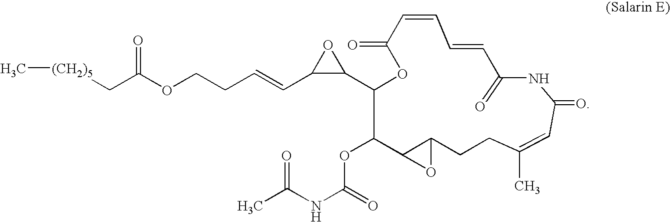

In some further embodiments, in a compound of general Formula I-A, R₃ and R₄ each independently is selected from null, —H, —OR₁₄, and —C(O)C₁-C₆-alkyl, as defined above. R₃ and R₄ may be different or same. R₁₄ is selected from —H and C₁-C₆ alkyl.

As used herein, the term “null” and the term “absent” are used interchangeably to designate the lacking of a specific group (e.g., R group), provided that the absence of a specific variant does not result in the formation of a charged atom. For example, in some embodiments, R₅ may be null. In such embodiments, the lone pair of electrons bonding said variant to the N atom forms a double bond with an adjacent atom, as disclosed herein. In other examples, a certain variant may be replaced by a hydrogen atom.

In some embodiments in a compound of general Formula I-A, R₃ is —OR₁₄ and R₄ is selected from —H and —C(O)C₁-C₆-alkyl. In these embodiments, R₅ and R₉ may optionally each be —H.

In some embodiments, R₃ is —OCH₃, and R₄ is —C(O)CH₃. In other embodiments, R₃ is —OCH₃, R₄ is —C(O)CH₃ and R₅ and R₉ are each —H, and the compound of the invention is herein designated Salarin D:

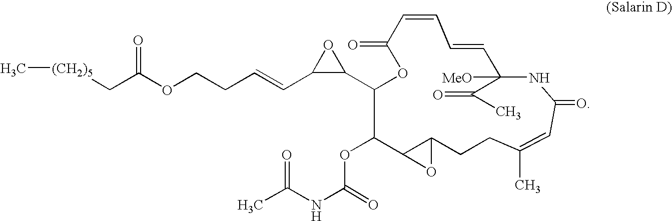

In a compound of general Formula I, R₁ and R₂ each independently is selected from —H, —OR₁₁ and —NR₁₂R₁₃, wherein R₁₁, R₁₂ and R₁₃ are as defined hereinabove.

In some embodiments, R₁ and R₂ are the same. In some other embodiments, R₁ is different from R₂.

In some embodiments, R₁ and R₂ are each —OH, the C₈-C₉ bond and the C₁₈-C₁₉ bond are each selected from a single bond and a double bond.

In some embodiments, the compound of general Formula I, is a compound of general Formula I-C:

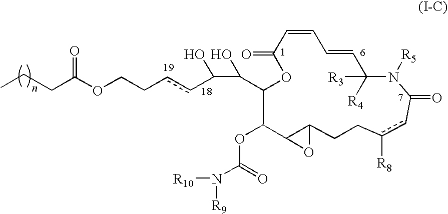

wherein R₃, R₄, R₅, R₈, R₉, R₁₀ and n are as defined hereinabove.

In some embodiments of a compound of general Formula I-C, the C₈-C₉ bond and the C₁₈-C₁₉ bond are each a double bond (each being cis or trans).

In some further embodiments, R₃ and R₄ each independently is selected from —H, —OR₁₄, and —C(O)C₁-C₆-alkyl, or R₃ and R₄ together with the carbon atom to which they are bonded form a carbonyl group.

In some additional embodiments, R₃ is —OR₁₄ and R₄ is selected from —H and —C(O)C₁-C₆-alkyl.

Optionally, in some embodiment R₅ is —H, R₁₄ is a methyl and R₄ is —C(O)C₁-C₆-alkyl, wherein the C₁-C₆-alkyl is selected from methyl, ethyl and propyl.

An example of a compound of Formula I-C is a compound herein designated Salarin B:

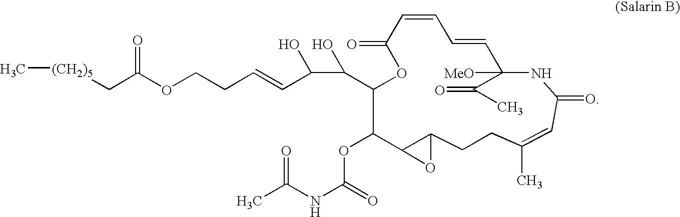

In some embodiments, in the compound of general Formula I-C, R₃ and R₄ together with the carbon atom to which they are bonded form a carbonyl group. In some additional embodiments, where R₃ and R₄ together with the carbon atom to which they are bonded form a carbonyl group, R₅ is —C(O)C₁-C₆-alkyl.

Another exemplary compound of general Formula I-C is a compound herein designated Salarin G:

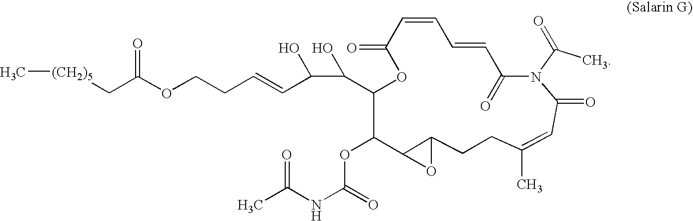

In some other embodiments, a compound according to the invention is a compound of general Formula I, wherein:

R₁ and R₂ each independently is selected from null, —H, —OR₁₁, and —NR₁₂R₁₃,

or

R₁ and R₂ together with the carbon atoms to which they are bonded form a heterocyclic ring system having 3 or 5 atoms, said heterocyclic ring comprising at least one heteroatom selected from O and N;

R₃ and R₄ together with the carbon atom C₆ to which they are bonded form the group C₆═CR₁₅R₁₆; R₁₅ and R₇ together with the carbon atoms to which they are bonded, form a 5-membered heterocyclic ring;

R₅ and R₆ are absent;

R₈ is selected from —H and C₁-C₆-alkyl;

R₉ is selected from —H, C₁-C₆-alkyl, C₁-C₆-alkylene-C₆-C₁₀-aryl and C₆-C₁₀-arylene-C₁-C₆-alkyl;

R₁₀ is selected from —H, C₁-C₆-alkyl and —C(O)R₁₇;

R₁₁ is selected from —H and C₁-C₆-alkyl;

R₁₂ and R₁₃ independently of each other is selected from —H and C₁-C₆-alkyl;

R₁₅ and R₁₆ each independently is selected from —H and C₁-C₆-alkyl;

R₁₇ is a C₁-C₆-alkyl;

n is an integer from 0 to 12;

the C₈-C₉ bond is a single or a double bond (cis or trans);

and

wherein the C₁₈-C₁₉ bond is a single or a double bond (cis or trans).

In some embodiments, the compound of the invention is a compound wherein the C₈-C₉ bond and the C₁₈-C₁₉ bond are each a double bond, being in the cis or trans configuration. In some embodiments, the C₈-C₉ bond is trans and the C₁₈-C₁₉ bond is cis. In other embodiments, the C₈-C₉ bond is cis and the C₁₈-C₁₉ bond is trans. In still other embodiments, both the C₈-C₉ bond and the C₁₈-C₁₉ are cis or both are trans.

In some other embodiments, said compound of general Formula I is a compound of the general Formula I-D:

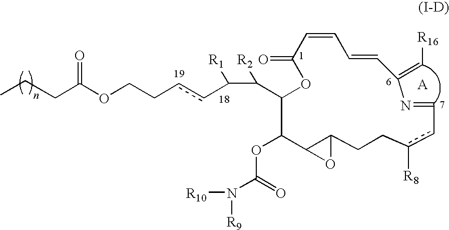

wherein each of R₁, R₂, R₈, R₉, R₁₀, R₁₆ and n are as defined hereinabove, and wherein the ring A:

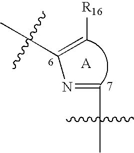

is a 5-membered ring having optionally one additional heteroatom selected from N and O. In some embodiments, the additional heteroatom is O.

In some embodiments in a compound of general Formula I-D, R₁ and R₂ together with the carbon atoms to which they are bonded form a 3- or 5-membered heterocyclic ring system comprising at least one heteroatom selected from O and N;

R₉ is selected from —H, C₁-C₆-alkyl, C₁-C₆-alkylene-C₆-C₁₀-aryl and C₆-C₁₀-arylene-C₁-C₆-alkyl;

R₁₀ is selected from —H, C₁-C₆-alkyl and —C(O)R₁₇;

R₁₅ and R₁₆ each independently is selected from —H and C₁-C₆-alkyl;

R₁₇ is a C₁-C₆-alkyl; and

wherein ring A is a 5-membered ring comprising one N atom and one O atom.

In some further embodiments, R₁ and R₂ together with the carbon atoms to which they are bonded form an epoxide ring, and the compound is a compound of general Formula I-E:

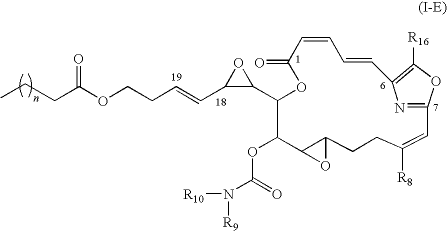

wherein each of R₈, R₉, R₁₀, R₁₆ and n are as defined hereinabove.

In some embodiments, in a compound of general Formula I-D or I-E:

R₈ is selected from —H and C₁-C₆-alkyl;

R₉ is selected from —H, C₁-C₆-alkyl, C₁-C₆-alkylene-C₆-C₁₀-aryl and C₆-C₁₀-arylene-C₁-C₆-alkyl;

R₁₀ is selected from —H, C₁-C₆-alkyl and —C(O)R₁₇;

R₁₆ is selected from —H and C₁-C₆-alkyl;

and

n is an integer from 0 to 12;

In some embodiments, in a compound of Formula I-E, R₁₆ is C₁-C₆-alkyl.

In other embodiments, R₉ is selected from —H, C₁-C₆-alkyl, C₁-C₆-alkylene-C₆-C₁₀-aryl and C₆-C₁₀-arylene-C₁-C₆-alkyl.

In further embodiments, R₁₆ is C₁-C₆-alkyl and R₉ is selected from —H, C₁-C₆-alkyl, C₁-C₆-alkylene-C₆-C₁₀-aryl and C₆-C₁₀-arylene-C₁-C₆-alkyl.

As used herein, “C₁-C₆-alkylene-C₆-C₁₀-aryl” refers to a carbon group, the alkylene, having between 1 and 6 carbon atoms, e.g., 1, 2, 3, 4, 5 or 6 carbon atoms, bonded on one end to a ring atom (in case of R₁₆) and to another end to an aromatic, e.g., aryl ring (monocyclic, bicyclic, tethered or fused) having between 6 and 10 carbon atoms, in some embodiments 6 or 10 carbon atoms. In some embodiments, the alkylene is selected from methylene (—CH₂—), ethylene (—CH₂CH₂—), propylene (—CH₂CH₂CH₂—), butylenes, etc., and said aryl being selected from a phenyl and a naphthyl group.

In some embodiments, the C₁-C₆-alkylene-C₆-C₁₀-aryl is a C₁-C₆-alkylene-phenyl, said phenyl residue being optionally substituted by one or more halide (e.g., Cl, Br, I, or F). In some embodiments, the phenyl ring is monosubstituted at the meta, ortho or para position. In further embodiments, the phenyl ring is disubstituted by two halides, being the same or different, at positions 1,2 or 2,3 or 3,4 or 1,3 or 1,4 or 1,5or 2,4 or 2,5 or 2,6, wherein position 1 is the ortho position to the alkylene, position 2 is the meta position, and position 3 is the para position to the alkylene group. In further embodiments, the phenyl is multisubstituted by three or more halide atoms, being the same or different.

As used herein, “C₆-C₁₀-arylene-C₁-C₆-alkyl” refers to an aromatic group, the arylene, having between 6 and 10 carbon atoms, in some embodiments 6 or 10 carbon atoms, bonded on one end to a ring atom (in case of R₁₆) and to another end to an alkyl, e.g., alkyl having 1, 2, 3, 4, 5 or 6 carbon atoms. In some embodiments, the aryl is phenylene or naphthylene and the alkyl is as defined above. In some embodiments, the arylene, e.g., phenylene is in the ortho, meta or para bonding configuration.

In other embodiments, R₉ is —H and R₁₆ is a methyl group, and the compound of general Formula I-E is of the general Formula I-F:

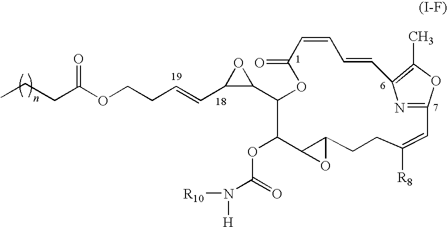

wherein;

R₈ is selected from —H and C₁-C₆-alkyl;

R₁₀ is selected from —H, C₁-C₆-alkyl and —C(O)R₁₇; wherein R₁₇ is as defined above; and

n is an integer from 0 to 12;

In additional embodiments, compounds of the invention include those in which R₁₀ is —C(O)C₁-C₆-alkyl. In some embodiments, said alkyl is a methyl group.

Thus, another example of a compound of the invention is a compound herein designated Salarin C:

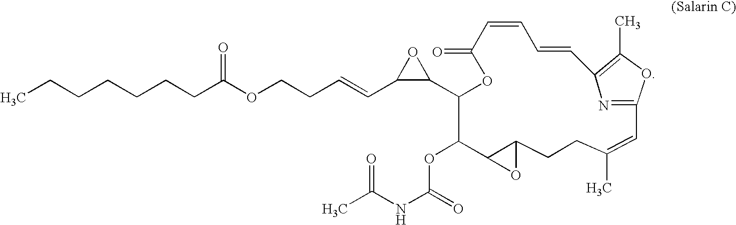

In some further embodiments, compounds of general Formula I-F include those in which R₉ is C₁-C₆-alkylene-C₆-C₁₀-aryl, as defined above, being optionally substituted by at least one halide and R₁₀ is —C(O)C₁-C₆-alkyl.

Optionally, the alkyl is a methyl and the aryl is a phenyl, being optionally substituted by at least one halide. An exemplary compound is a compound herein designated Salarin C-5:

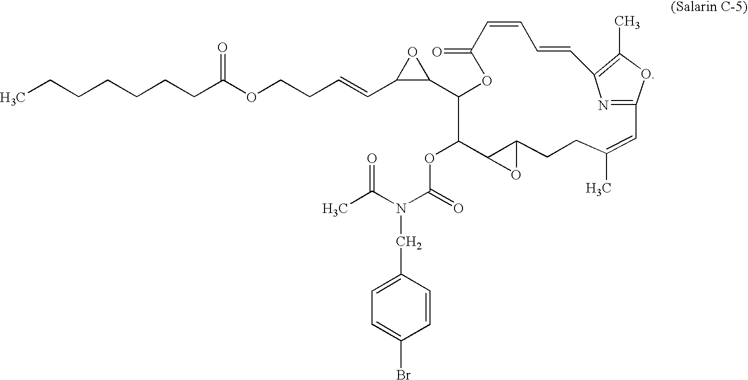

In further embodiments of compounds of general Formula I-F, compounds of the invention include those in which R₁₀ is —H, said compounds being exemplified by a compound herein designated Salarin C-4:

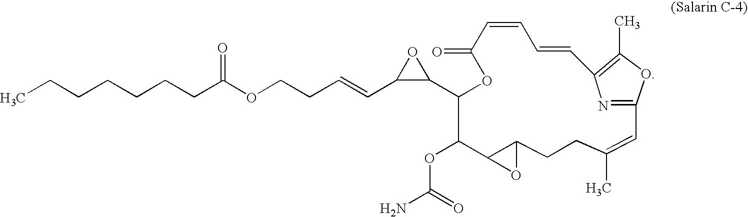

In further embodiments, compounds of the general Formula I-D are those in which R₁ and R₂ each independently is selected from null, —H, —OR₁₁, and —NR₁₂R₁₃.

In some embodiments, each of R₁ and R₂ may be —H or —OH.

Another compound according to the invention, being a representative of compounds of general Formula I-D is a compound herein designated Salarin F:

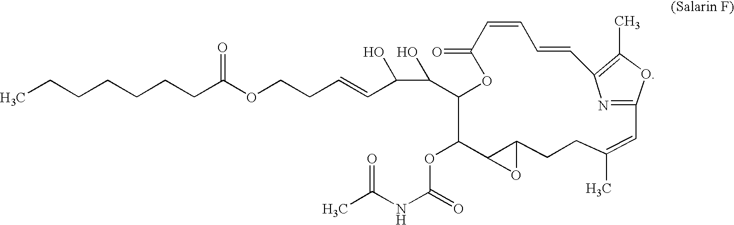

In some embodiments, in a compound of general Formula I-D, R₁ is —OH and R₂ is —H and one or both of the C₈-C₉ bond and the C₁₈-C₁₉ bond is a single bond.

In some embodiments, the C₈-C₉ bond and the C₁₈-C₁₉ bond are each a single bond and an exemplary compound is a compound herein designated Salarin C-3:

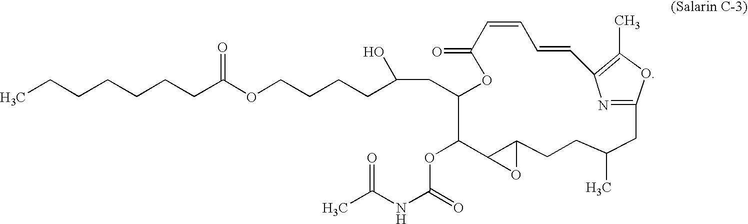

In some further embodiments, compounds of the general Formula I-D are those in which R₁ and R₂ form a heterocyclic ring system, said ring is a 5-membered ring comprising at least one oxygen atom, said 5-membered ring being of the general formula:

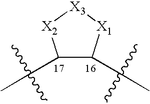

wherein at least one of X₁, X₂ and X₃ is —O— and the others of X₁, X₂ and X₃ are as defined hereinabove.

In some embodiments, X₁ and X₂ are —O— and X₃ is selected from CH₂, CHhal, C(hal)₂, CH(C₁-C₆-alkyl), C(C₁-C₆-alkyl)₂, CH(C₆-C₁₀-aryl) and C(C₆-C₁₀-aryl)₂.

In further embodiments, X₁ and X₂ are —O— and X₃ is selected from CH₂, CH(C₁-C₆-alkyl), C(C₁-C₆-alkyl)₂, CH(C₆-C₁₀-aryl) and C(C₆-C₁₀-aryl)₂.

In additional embodiments, X₁ and X₂ are —O— and X₃ is selected from CH₂, CH(C₁-C₆-alkyl) and C(C₁-C₆-alkyl)₂.

Still further, Xᵢ and X₂ are —O— and X₃ is C(C₁-C₆-alkyl)₂. In some embodiments, where X₃ is C(C₁-C₆-alkyl)₂, the two C₁-C₆-alkyl groups are different or same, selected from methyl, ethyl and propyl.

One such compound is a compound herein designated Salarin C-2:

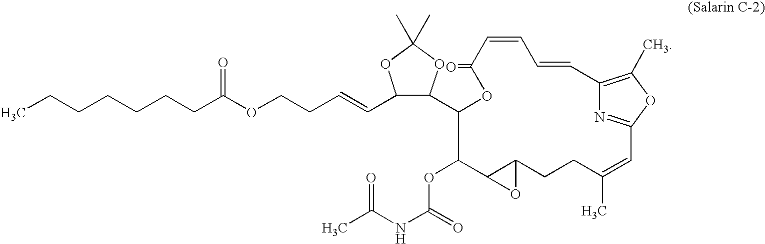

In another aspect of the present invention there is provided a compound of the general Formula II, including isomers, e.g., stereoisomers, geometrical isomers; solvates; and salts thereof, whether pharmaceutically acceptable salts or otherwise:

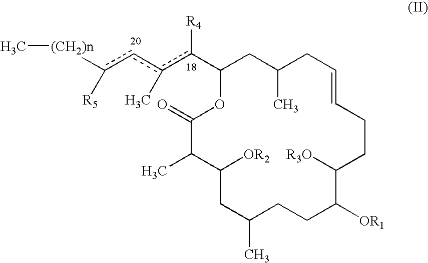

wherein,

R₁ is selected from —H, C₁-C₆-alkyl and —C(O)NR₆R₇;

R₂ and R₃ each independently is selected from —H, -Ts, —C(O)NR₈R₉, —C(O)—C₁-C₆-alkyl and —C(O)—C₆-C₁₀-aryl;

R₄ is selected from —H, —O—, —OR₁₀, —N—, and —NR₁₁R₁₂; and R₅ is selected from —H, —OR₁₃, —N—, and —NR₁₄R₁₅

or

R₄ and R₅ together with the carbon atoms to which they are bonded form a heterocyclic ring system comprising at least one heteroatom selected from N and O; said ring system being a monocyclic or a multicyclic system; wherein each of said R₁₀, R₁₁, R₁₂, R₁₃, R₁₄ and R₁₅ is optionally a bond or an atom of said ring system or independently selected from —H and C₁-C₆-alkyl;

R₆, R₇, R₈ and R₉ each independently is selected from —H and C₁-C₆-alkyl;

n is an integer from 0-6 (i.e., 0, 1, 2, 3, 4, 5, or 6);

the C₁₈-C₁₉ bond is a single bond or a double bond;

the C₁₉-C₂₀ bond is a single bond or a double bond; and

the C₂₀-C₂₁ bond is a single bond or a double bond.

As used herein, “Ts” refers to a tosyl group.

In some embodiments, in a compound of general Formula II:

R₁ is selected from —H, C₁-C₆-alkyl and —C(O)NR₆R₇;

R₂ is selected from —H, -Ts, —C(O)—C₁-C₆-alkyl; —R₃ is selected from —H, -Ts, —C(O)NR₈R₉ and —C(O)—C₁-C₆-alkyl;

R₄ and R₅ are each —H;

R₆, R₇, R₈ and R₉ each independently is selected from —H and C₁-C₆-alkyl;

n is an integer from 0-6;

the C₁₈-C₁₉ bond is a single bond or a double bond;

the C₁₉-C₂₀ bond is a single bond; and

wherein the C₂₀-C₂₁ bond is a single or a double bond.

In further embodiments, in a compound of general Formula II the C₁₈-C₁₉ bond and the C₂₀-C₂₁ bond are each a double bond.

In some further embodiments, R₁ is —C(O)NR₆R₇ and the compound is a compound of the general Formula II-A:

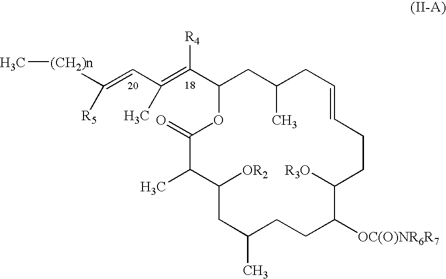

wherein R₂, R₃, R₄, R₅, R₆ and R₇ are as defined hereinabove.

In some embodiments, R₄ and R₅ are each —H.

In some embodiments, R₂ and R₃ are each —H. In further embodiments, one of R₂ and R₃ is —H and the other is different from —H. In some embodiments, R₃ is —H and R₂ is not. In other embodiments, R₂ is —H and R₃ is not.

A non-limiting example of a compound according to Formula II-A is the compound herein designated Tulearin A:

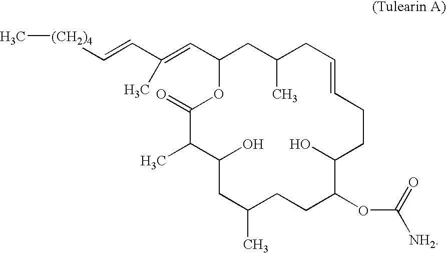

In some embodiments, compounds of the invention include those in which one of R₂ and R₃ of Formula II is —H. An example of such a compound wherein R₃ is —H, is a compound herein designated Tulearin B:

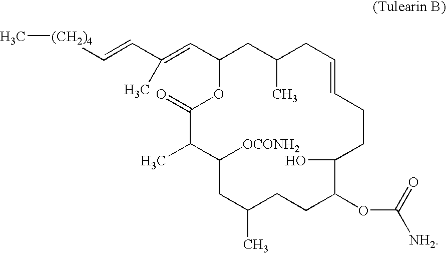

In the following compound, R₂ is —H, said compound being designated Tulearin A-3:

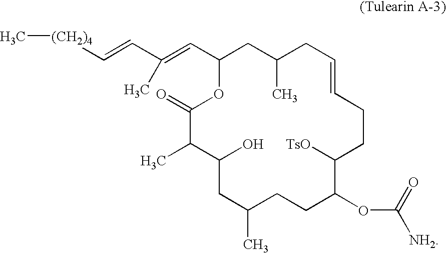

Another example of a compound of general Formula II-A, wherein R₂ is —H, is a compound of the general Formula II-B:

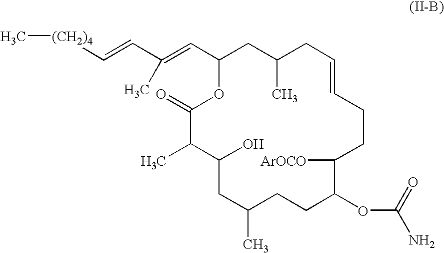

wherein Ar is an aryl, e.g., phenyl substituted by at least one halide atom selected from Cl, Br, I and F, said substitution is as defined hereinabove.

In some embodiments of a compound of general Formula II-B, where Ar is p-bromobenzene, the compound is herein designated Tulearin A-4:

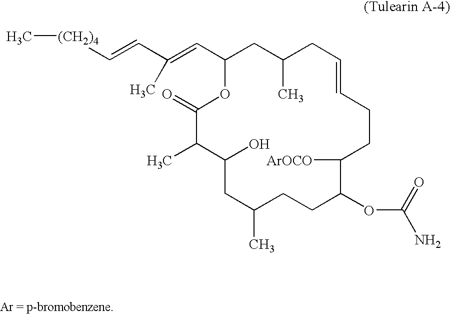

In further embodiments, the compound of Formula II-A is a compound wherein both R₂ and R₃ are different from —H. An exemplary compound is a compound herein designated Tulearin A-5:

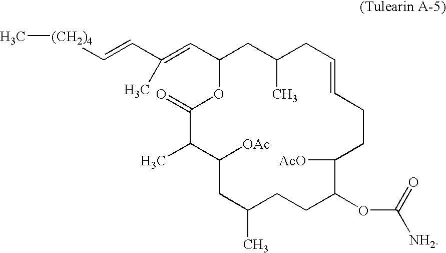

In some further embodiments of a compound of general Formula II or Formula II-A:

R₁ is selected from —H, C₁-C₆-alkyl and —C(O)NR₆R₇;

R₂ is selected from —H, -Ts, —C(O)—C₁-C₆-alkyl;

R₃ is selected from —H, -Ts, —C(O)NR₉R₁₀ and —C(O)—C₁-C₆-alkyl;

R₄ and R₅ together with the carbon atoms to which they are bonded form a heterocyclic ring system comprising at least one heteroatom selected from N and O; said ring system being a monocyclic or a multicyclic system;

R₆, R₇, R₈ and R₉ each independently is selected from —H and C₁-C₆-alkyl;

n is 0 or an integer from 1-6;

the C₁₈-C₁₉ bond and the C₂₀-C₂₁ bond are each a single bond; and

the bond C₁₉-C₂₀ is a double bond.

In some embodiments, R₄ and R₅ together with the carbon atoms to which they are bonded form a heterocyclic ring system comprising at least one N atom; said ring system being a monocyclic system (i.e., a non-aromatic heterocycle having a single ring), optionally substituted by at least one C₁-C₆-alkyl, C₆-C₁₀-aryl, and at least one halide.

The monocyclic ring is a 6-membered ring of the formula:

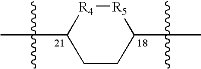

wherein R₄ is —NR₁₁; and R₅ is —NR₁₄, wherein each of R₁₁ and R₁₄ independently selected from a bond, —H and C₁-C₆-alkyl. In some embodiments, the 6-membered ring is thus of the general formula:

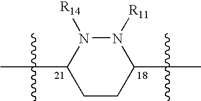

wherein each of R₁₁ and R₁₄ is a C₁-C₆-alkyl. In some embodiments, R₁₁ and R₁₄ together with the N atoms to which they are bonded, form a multicyclic system (i.e., having two or more non-aromatic heterocyclic rings being fused or tethered to each other via one or more covalent bonds) said ring system being optionally substituted by at least one C₁-C₆-alkyl, C₆-C₁₀-aryl, and at least one halide. In some embodiments, said ring system may be oxidized (e.g., have at least one sp² hybridized carbon atom such as endo- or exocyclic C═C or C═O carbonyl groups)

As shown above, in some embodiments, said multicyclic ring system is a two-ring fused system, being optionally substituted, and being generally of the formula:

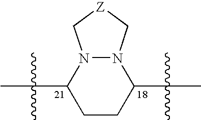

wherein Z is selected from —CH₂—, —CH(C₁-C₆-alkyl), —C(C₁-C₆-alkyl)₂, —CH(C₆-C₁₀-aryl), —C(C₆-C₁₀-aryl)₂, —CH₂CH₂—, —NH, —N—C₁-C₆-alkyl, —N—C₆-C₁₀-aryl and —O—.

In some embodiments, the fused ring system is of the formula:

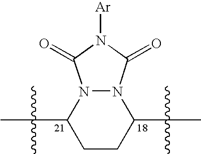

wherein Ar is a C₆-C₁₀-aryl selected from phenyl and naphthyl being optionally substituted by at least one group selected from an halide and a C₁-C₆-alkyl.

The invention thus provides, as an example of a compound of general Formula II or II-A, a compound herein designated Tulearin A-1 and Tulearin A-2:

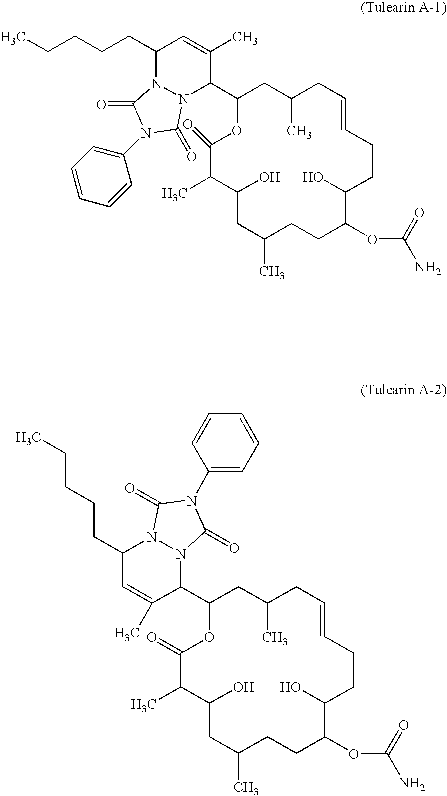

In some embodiments of a compound of general Formula II, II-A or II-B, both R₂ and R₃ are -Ts.

As used herein, a compound according to the present invention is any one compound of the general Formulae I, I-A, I-B, I-C, I-D, I-E, I-F, II, II-A and II-B, including compounds herein specifically designated and any compound falling within the scope of the invention as claimed.

In another aspect of the present invention, there is provided a compound being selected amongst compounds herein designated: Salarin A, Salarin A-2, Salarin E, Salarin D, Salarin B, Salarin G, Salarin C, Salarin C-5, Salarin C-4, Salarin F, Salarin C-3, Salarin C-2, Tulearin A, Tulearin B, Tulearin A-3, Tulearin A-4, Tulearin A-5, Tulearin A-1, and Tulearin A-2, any isomers thereof and salts thereof, whether isolated from natural sources or synthetically or semi-synthetically (based on isolated parent compounds) manufactured.

In another aspect of the present invention, there is provided a compound having the spectroscopy data (NMR, IR, UV, MS, etc) provided in any of the Tables, Figures and in of the examples provided herein.

As a person skilled in the art would realize, the compounds of the invention contain one or more chiral centers. Such chiral centers may be of either the (R) or (S) configuration, or may be a mixture thereof. The invention therefore also provides any one compound according to the invention in an enantiomerically pure form, or in any one mixture form, whether stereoisomeric or diastereomeric.

Where the herein-described processes for the isolation/preparation of the compounds give rise to mixtures of stereoisomers, these isomers may be separated by conventional techniques, such as preparative chromatography. The compounds may be prepared in racemic form, or individual enantiomers may be prepared either by enantiospecific synthesis or by chiral chromatographic separation of a racemate.

The invention thus also provides in another of its aspect, a mixture of enantiomers, stereoisomers and diasteriomers of each of the compounds of general Formula I and II and any sub-structure. In one example, the invention provides a Tulearin A-1 mixture comprising all enantiomers, stereoisomers and diasteriomers, whether isolated in pure or as a mixture of the compound. Such a mixture will, in some embodiments, comprise also Tulearin A-2.

The compounds of the invention may be obtained by either isolation of compounds such as those herein designated as Salarin A, Salarin E, Salarin D, Salarin B, Salarin G, Salarin C, Salarin F, Tulearin A and Tulearin B or by chemical modification of the isolated compounds, employing one or more functional groups modification or transformation of the parent compound or any compound derived therefrom to provide such compounds as herein designated Salarin A-2, Salarin C-5, Salarin C-4, Salarin C-3, Salarin C-2, Tulearin A-3, Tulearin A-4, Tulearin A-5, Tulearin A-1 and Tulearin A-2. In some cases, one or more of the isolated compounds may be converted, as disclosed herein, to one or more compounds according to the invention.

The present invention thus also contemplates derivatives of any compound of the present invention. The term “derivative” as used herein refers to a chemically modified compound derived from a parent compound of the invention (e.g., Salarin A, Salarin B, Salarin C or Tulearin A, etc) that differs from the parent compound by one or more elements, substituents and/or functional groups such that the derivative has the same or similar biological properties/activities as the parent compound, as defined herein.

In some embodiments, the derivative is obtained by substitution of at least one nucleophilic atom, e.g., a nitrogen atom.

In some embodiments, the derivative is obtained by intramolecular or intermolecular rearrangement.

In further embodiments, the derivative is obtained by a condensation reaction.

In still other embodiments, the derivative is obtained utilizing a Diels-Alder reaction.

Non-limiting examples of derivatives obtained from the parent salarines and tulearins are ester, amide, or carbamate esters, including prodrug forms thereof which upon administration to a subject are capable of providing (such as by metabolic process) a compound of the present invention or an active metabolite thereof. As used herein, the derivatives of the invention maintain the biological activity of the parent compounds.

It should be appreciated that the compounds disclosed herein encompass also solvates thereof. As used herein, the term “solvate” refers to a complex of variable stoichiometry formed by a solute and a solvent. Such solvents for the purpose of the invention may not interfere with the biological activity of the solute. Examples of suitable solvents include, but are not limited to, water, methanol and ethanol.

The compounds of the present invention can be formulated into a composition, preferably a pharmaceutical composition, as, e.g., neutralized pharmaceutically acceptable salt form. Pharmaceutically acceptable salts include the acid addition salts (formed with the free amino groups of a compound of the invention), which are formed with inorganic acids such as hydrochloric or phosphoric acids, or such organic acids as acetic, oxalic, tartaric, mandelic, and the like. Salts formed from the free hydroxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Some of the compounds of the invention are acidic and they form salts with a pharmaceutical acceptable cation. Some of the compounds of this invention are basic and form salts with pharmaceutical acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

The compounds of the present invention may additionally, or alternatively, form salts of pharmaceutically unacceptable cations or anions for various other purposes such as non-medical purposes.

In another aspect of the present invention there is provided a use of at least one compound according to the invention for the preparation of a composition.

In some embodiments, the composition is a pharmaceutical composition. The pharmaceutical composition may be employed in a therapeutic regimen for the treatment or prevention of a disease or disorder.

In some embodiments, said disease or disorder is a hyperproliferative disease or disorder. In additional embodiments, such disease or disorder is cancer.

The invention, thus, also provides compositions and pharmaceutical compositions comprising one or more compound according to the invention.

The composition of the invention may comprise a single compound of the invention or two or more such compounds. In some embodiments, the composition comprises a single compound of the invention presented in two different forms, e.g., in a free base form and in a salt form. Alternatively, the composition may comprise two different salts, stereoisomers, geometrical isomers, solvates, etc., of a compound according to the invention.

In some embodiments, the composition, particularly the pharmaceutical composition comprises at least one pharmaceutically acceptable carrier, excipient or diluent.

As used herein, “pharmaceutical composition” means therapeutically effective amounts of a compound of the present invention, together with a suitable carrier. Such compositions are liquids or lyophilized or otherwise solid formulations. The pharmaceutical composition is suitable for administration in any one methods known in the art, such as parenterally, paracancerally, transmucosally, transdermally, intramuscularly, intravenously, intradermally, subcutaneously, intraperitonealy, intraventricularly, intracranially and intratumorally.

In some embodiments, the compositions of the invention are for oral administration.

The pharmaceutically acceptable carrier is, for example, a vehicle, an adjuvant, an excipient, and/or a diluent. The carrier is one which is chemically inert to the active compound comprised in the composition and one which has no detrimental side effects or toxicity under the conditions of use.

The choice of carrier will be determined in part by the particular active compound, as well as by the particular method used to administer the composition. Accordingly, there is a wide variety of suitable formulations of the pharmaceutical composition of the present invention. The following formulations are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active compound, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active compound in a flavor, usually sucrose and acacia or tragacanth, as well as pastilles comprising the active compound in an inert base, such as gelatin and glycerin, or sucrose and acacia, emulsions, gels, and the like containing, in addition to the active compound, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxy-methylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

The parenteral formulations will typically contain from about 0.5 to about 25% by weight of the active compound in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active compound, such carriers as are known in the art to be appropriate.

Also contemplated by the invention are particulate compositions coated with polymers (e.g., poloxamers or poloxamines). Other embodiments of the compositions of the invention incorporate particulate forms, protective coatings, protease inhibitors or permeation enhancers for various routes of administration, including parenteral, pulmonary, nasal and oral.

In yet other embodiments, the pharmaceutical composition can be delivered in a controlled or a sustained release system. For example, the composition may be administered using intravenous infusion, an implantable osmotic pump, a transdermal patch, liposomes, or other modes of administration.

In another embodiment, polymeric materials can be used.

The compositions of the invention comprising one or more compound according to the present invention may be co-administered with an ongoing therapy (e.g., medicament) or may be prescribed by a medical practitioner in combination with other medicaments or modalities of treatment. In some embodiments, the composition of the invention is administered along with chemotherapy, radiotherapy, or another treatment, e.g., cancer treatment.

The dose of a compound of the present invention in the pharmaceutical composition may be appropriately set or adjusted in accordance with an administration form, an administration route, a degree or stage of a target disease, and other parameters. Dosing may be in one or a combination of two or more administrations, e.g., daily, bi-daily, weekly, monthly, or otherwise in accordance with the judgment of the clinician or practitioner, taking into account factors such as age, weight, severity of the disease, and the dose administered in each administration.

The pharmaceutical compositions of the present invention are suitable for use in medicine. In some embodiments, the compositions are for the treatment and/or prophylaxis of at least one disease or disorder. In other embodiments, the disease or disorder is associated with cell hyperproliferation.

Thus, the invention further provides a method for the treatment and/or prevention of a disease or disorder, said method comprising administering to a subject (e.g., a human or non-human animal) suffering from or having predisposition to suffer from or having symptoms associated with said disease or disorder, an effective amount of a compound according to the present invention or a composition comprising thereof.

In some embodiments, said disease or disorder is associated with cell hyperproliferation.

As used herein, “hyperproliferation” refers to a disease state in which cells grow in an uncontrolled manner, whether that growth is cancerous or not. The term also encompasses cell growth that is independent of normal regulatory mechanisms (e.g., loss of contact inhibition).

In some embodiments, said disease or disorder is cancer. Non-limiting examples of cancer are:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostrate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfrorna (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningio sarcoma, gliomatosis), brain (astrocytoma, medulloblastoma, glioma, ependymoma, germinoma, glioblastoma multiform, oligodendroglioma, schwannoma, retinoblastoma, congenital tumors), spinal cord (neurofibroma, meningioma, glioma, sarcoma);

Gynecological: uterus (endometrial carcinoma), cervix (cervical carcinoma, pre-tumor cervical dysplasia), ovaries (ovarian carcinoma, granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma), fallopian tubes (carcinoma);

Hematologic: blood (acute and chronic myeloid leukemia, acute lymphoblastic leukemia, chronic lymphocytic leukemia, multiple myeloma, myelodysplastic syndrome), Hodgkin's disease, non-Hodgkin's lymphoma;

Skin: malignant melanoma, basal cell carcinoma, squamous cell carcinoma, Karposi's sarcoma, moles dysplastic nevi, lipoma, angioma, dermatofibroma, keloids, psoriasis; and cancers of the adrenal glands such as neuroblastoma.

The disease or disorder is also selected amongst myeloproliferative diseases such as polycythemia vera, primary myelofibrosis, thrombocythemia, essential thrombocythemia, agnoneic myeloid metaplasia, leukemia, chronic myelogenous leukemia, systemic mastocystosis, chronic neutrophilic leukemia, myelodisplastic syndrome and systemic mast cell disease.

In some embodiments, said cancer is leukemia.

The invention also provides a method for the treatment of cancer, said method comprising administering to a subject suffering form cancer a therapeutically effective amount of a compound according to the invention.

In another aspect of the present invention there is provided the use in medicine of a compound according to the invention. In some embodiment the use is for the treatment or prevention of cancer.

As used herein, the term “treatment”, or any lingual variation thereof refers to the administering of a therapeutically effective amount of the composition of the present invention which is effective to ameliorate undesired symptoms associated with a disease, e.g., cancer, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring spreading in the body or a combination of two or more of the above.

The term also includes the eradication, removal, modification, or control of primary, regional, or metastatic cancer tissue; and the minimizing or delay of the spread (metastasis) of the cancer.

The “effective amount” for purposes herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

Specifically, a composition of the invention should be administered in an effective amount sufficient to destroy, modify, control or remove a primary, regional or metastatic cancer cell or tissue; delay or minimize the spread of cancer; or provide a therapeutic benefit in the treatment or management of cancer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein, the singular form “a”, “an” and “the” include plural references unless the context clearly dictates otherwise. For example, the term “a compound” or “at least one compound” may independently include a plurality of compounds, including mixtures thereof.

It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range. For example, an alkyl chain having between 1 and 6 carbon atoms should be considered to have specifically disclosed sub-ranges such as from 1 to 5, from 1 to 4, from 1 to 3, from 2 to 6, from 3 to 6, from 4 to 6, and from 4 to 6, as well as individual numbers within that range, for example, 1, 2, 3, 4, 5, and 6.

Various embodiments and aspects of the present invention as delineated hereinabove and as claimed in the claims section below find experimental support in the following examples.

### DETAILED DESCRIPTION OF THE INVENTION

The compounds herein designated as Salarin A, Salarin E, Salarin D, Salarin B, Salarin G, Salarin C, Salarin F, Tulearin A and Tulearin B have been isolated from the sponge Fascaplysinopsis sp., typically native to the Salary bay north to Tuléar, Madagascar, according to a procedure disclosed hereinbelow. Generally, samples of the sponge were extracted in an organic solvent or a mixture thereof and the resulting extract was subject to partitioning.

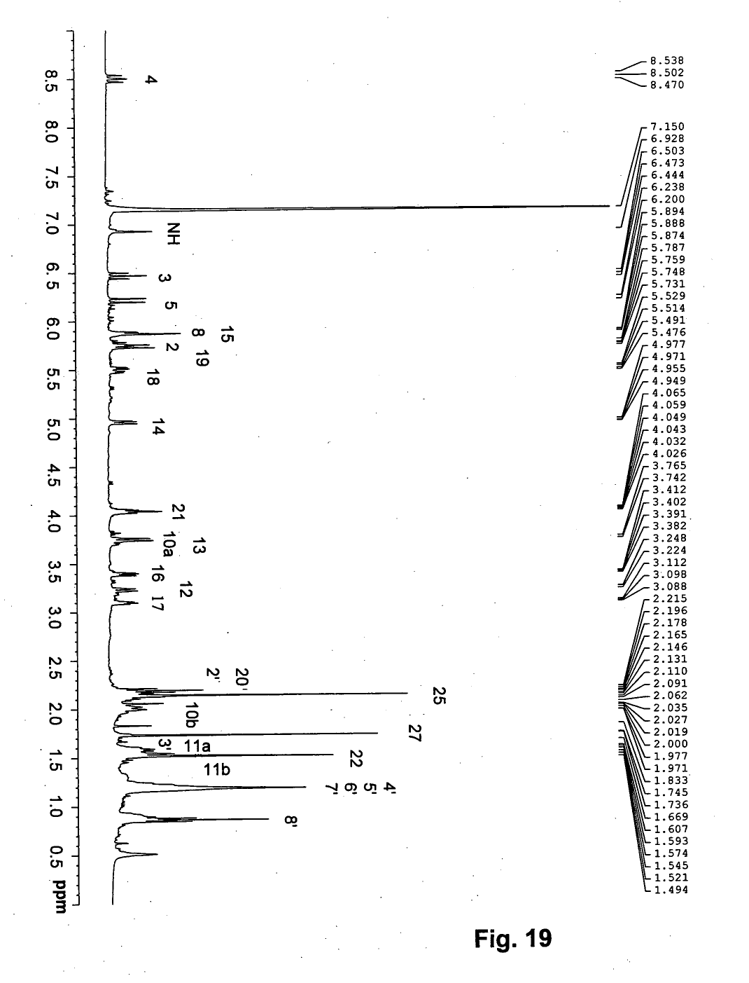

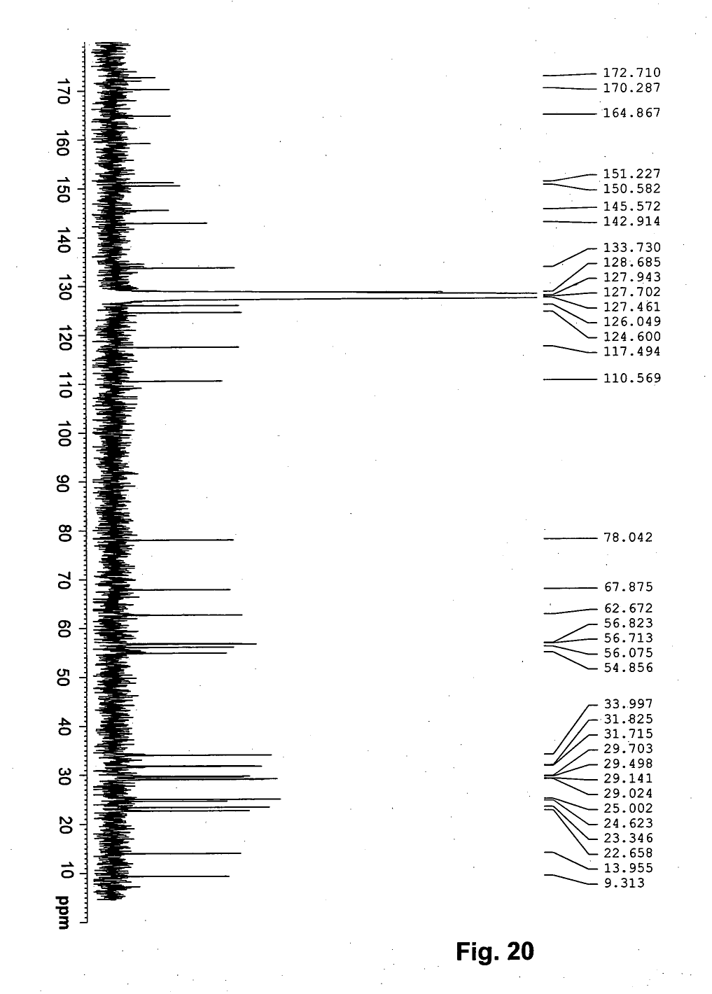

The Salarins described herein possess unique macrolide structures and unique acetylcarbamate groups. The uniqueness of the macrolide structure resides not only in the triacylamine and the substituted lactam functionalites, of Salarin A and Salarin B, respectively, but also in the construction of the macrolide from two carbon chains (a 6-aminohexa-2,4-dienoic acid and a functionalized C 15-acid). A combination of the two chains is rarely found in the nature, for example in the two nitrogenous macrolides madangolide [2,3] isolated from the cyanobacteria Lyngbia bouillonii. Without wishing to be bound by theory, it may be suggested that in Salarin C, the oxazole ring is biosynthesized from an α-acetyl ketoxime as depicted in FIG. 19. It may also be suggested that Salarins A and B are biosynthetically connected to Salarin C, as schematically shown in FIG. 20. Surprisingly, stirring Salarin C in chloroform slowly afforded (ca. 50% in 3 days) Salarin A. Isomerization of the αβ, γδ-dienoate may also be achieved.

The isolation process afforded mixtures of compounds which have been separated and characterized each individually according to methods known in the art. The isolated compounds described herein served as parent compounds for the synthesis of a great variety of derivatives, as disclosed herein. The derivatives were produced following synthetic routes functional group transformations. Purity of the isolated compounds and the derivatives thereof was for example determined by standard methods of analysis, such as thin layer chromatography (TLC), gel electrophoresis, high performance liquid chromatography (HPLC) and mass spectrometry (MS).

The following examples are not in any way intended to limit the scope of the inventions as claimed.

### EXAMPLES

### A. General Experimental Procedures

Optical rotations were obtained with a Jasco P-1010 polarimeter. IR spectra were obtained with a Bruker FTIR Vector 22 spectrometer. ¹H and ¹³C NMR spectra were recorded on Bruker Avance-500 spectrometers. COSY, HMQC, NOESY and HMBC were recorded using standard Bruker pulse sequences. FABMS measurements were recorded on a Fisons, Autospec Q instrument. MALDI (HRMS) measurements were recorded on Applied Biosystem Voyager DE-STR MALDI TOF instrument. Electrospray MS measurements were performed on an Applied Biosystems Q-STAR Pulsar instrument (ESI-QqTOF).

### B. Extraction and Isolation

Isolation of the compounds of the invention was guided by performing brine shrimp tests on all extracts and separated fractions. The test involved addition of few micrograms of the extract or compound in DMSO to a test tube containing 10 brine shrimp eggs and observation of the lethality of the embryos after 24 hours.

The frozen sponge Fascaplysinopsis sp. (110 g) collected in Salary bay north to Tuléar, Madagascar, was homogenized and extracted twice with CHCl₃/MeOH (2:1). The combined organic extract was concentrated to yield a crude extract (2.1 g) that was subjected to partitioning by the Kupchan method. The method involved partition between aqueous methanol (MeOH) and Hexane followed by partition between the remaining aqueous MeOH phase and carbon tetracloride (CCl₄) and partition between the remaining aqueous MeOH and dichloromethane (DCM) (CH₂Cl₂). Evaporation of the various phases resulted in 4 fractions according to their polarity. Part of dichloromethane fraction, 197 mg, was repeatedly chromatographed on a Sephadex LH-20 column, eluting with n-hexane/MeOH/CHCl₃ (2:1:1) to obtain 12 fractions of ca, 20 mL. Fractions 8-9 (22.5 mg) were chromatographed over silica gel (VLC) using n-hexane with increasing proportions of ethyl acetate as eluent. Salarin A (5.5 mg, 0.016 wt %) was afforded by elution with 40% ethyl acetate in hexane. Fractions 11-12 (12 mg) mainly containing Tulearin A were further purified by VLC over silica gel, eluted with 50% ethyl acetate in hexane to afford pure Tulearin A, (6.6 mg 0.019 wt %). Another collection of the sponge afforded in a similar way to the isolation of Salarin A, Salarin B (2.5 mg, 0.008 wt %). From yet another collection of the sponge, several additional compounds were isolated, designated as Salarin C, Salarin D, Salarin E, Salarin F, Salarin G, and Tulearin B.

### C. Synthetic derivatives of Salarin and Tulearin

The Salarin and Tulearin compounds isolated as described herein above were used as parent compounds for the synthesis of various derivatives. The derivatives were produced according to procedures well known in the art. All reactions were done on a 10 mg scale. After the reaction was completed, as was monitored by TLC, the solvent was evaporated under vacuum and the product was purified by chromatography (VLC) on silica gel. Specified percentages (%) are given in w/w (weight per weight), unless otherwise indicated.

### Synthesis of Salarin C-1

Salarin C was treated with cerium ammonium nitrate (CAN) in a mixture of acetonitrile (ACN) and water [CH₃CN/H₂O (5 mL)] for 3 hrs at room temperature (RT) to yield Salarin C-1 at a 20% calculated yield.

### Synthesis of Salarin C-2 and Salarin A-1

Salarin C was treated in acetone (An) (5 mL) with one drop of 7% HClO₄ at −20° C. for 10 min to yield a mixture of Salarin C-2 (with an acetonide group on C₁₆ and C₁₇) and Salarin A-1 at a 15% calculated yield each.

### Synthesis of Salarin C-5

Salarin C was reacted with p-bromobenzylbromide (BrCH₂C₆H₄Br) in acetone (5 mL) in the presence of 10 mg of K₂CO₃ for 48 hr at RT to yield the corresponding N-p-Bromobenzyl derivative Salarin C-5 at a 60% calculated yield.

### Synthesis of Salarin C-3

Salarin C was hydrogenated for 30 min in EtOH with 5% Pd on carbon at 1 atmosphere pressure. Salarin C-3 was obtained in 80% yield.

### Synthesis of Salarin C-4

Salarin C was hydrolyzed in 10% NH₄OH in MeOH, over night at RT to yield the deacetyl derivative Salarin C-4 at a 40% calculated yield.

### Synthesis of Salarin A-2

Salarin A (10 mg), K₂CO₃ (10 mg) and one drop of MeI, in acetone (5 ml) were kept at room temperature for 48 hrs. The sample was then evaporated filtered through a micro-column of silica gel to afford the N—CH₃ derivative Salarin A-2.

Similarly, Salarin A-2 was afforded by N-Methylation of Salarin C as p-bromobenzylation.

### Synthesis of Tulearin A-1 and Tulearin A-2

Tulearin A was reacted with N-phenyltriazolinedione in 5 ml dichloromethane (DCM) over night at R.T. The dienophile, N-phenyltriazolinedione, can approach the molecule from both sides, hence the reaction yielded the two diastereomers Tulearin A-1 and Tulearin A-2 at approximately 1:1 ratio. The diastereomers were separated on a silica gel column, eluted with petrol ether and ethyl acetate (EtOAc).

### Synthesis of Tulearin A-3

Tulearin A (10 mg) in pyridine (1 mL) was treated with toluenesulfonyl chloride (TsCl) (5 ml) at 0° C. for 48 hr. The reaction mixture was evaporated and chromatography through a silica gel column eluted with Hexane;EtOAc mixtures afforded the mono-substituted product Tulearin A-3 and the di-substituted product at a 4:1 ratio and 5% calculated total yield.

### Synthesis of Tulearin A-4

Tulearin A (10 mg) in dichloromethane (1 ml) was treated over night at RT with p-bromobenzoyl chloride (5 mg) at the presence of 5% (v/v) triethylamine (TEA). The reaction mixture was then evaporated and chromatography through a silica gel column afforded the benzoate Tulearin A-4 at a calculated 50% yield.

### Synthesis of Tulearin A-5

Tulearin A was acetylated in acetic anhydride:pyridine (Ac₂O):(Py) (1:1) (1 mL) at R.T over night. The reaction mixture was evaporated and the di-acetate Tulearin A-5 was obtained at a calculated 80% yield.

### D. Physical and Chemical Properties of the Various Compounds

### Salarin A:

Pale yellow oil; [α]²³D −57 (c 0.37, CHCl₃): IR (CHCl₃) νₘₐₓ 3690, 3028, 3010, 1728, 1602, 1370 cm⁻. ¹H and ¹³C NMR see Table 1. The mass spectrometric analysis of Salarin A provided a molecular formula of C₃₅H₄₆N₂O₁₂ (HRESMS m/z 709.2991 for [M+Na]⁺), with 14 degrees of unsaturation: HRMS— MALDI m/z 709.2991 [M+Na]⁺ (Calcd. for, C₃₅H₄₆N₂O₁₂Na, 709.2943). (negative) FABMS m/z 686 [M−H]⁻ (100), 643 ([M−H]-C₂H₃O) (10); 601 ([M−H]C₃H₃O₂N) (40), 558 ([M−H]C₅H₆O₃N) (10).

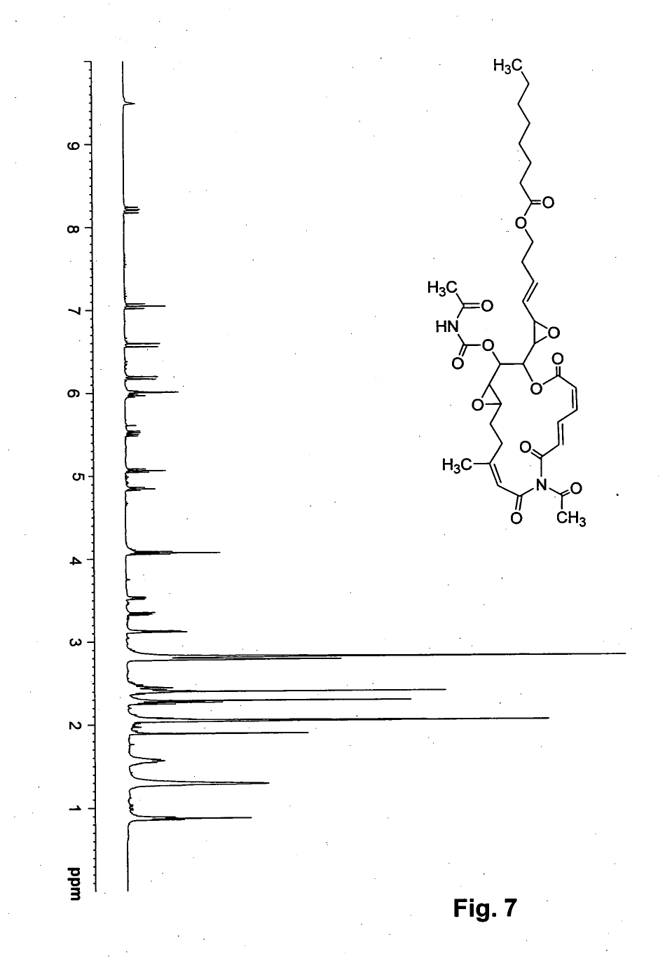

¹H-δvalues (500 MHz, Acetone-d₆): 9.48 s, 8.21 d, 7.05 t, 6.58 d, 6.18 d, 6.0 s, 5.98 dt, 5.52 dd, 5.06 t, 4.48 t, 4.08 t, 3.53 dd, 3.34 dd, 3.13 m, 3.12 m, 2.48 m, 2.39 s, 2.37 q, 2.30 s, 2.28 t, 2.04 m, 1.90 s, 1.58 m, 1.55 m, 1.30 m, 1.29 m, 1.28 m, 0.87 t. (¹H-NMR spectrum of Salarin A is showed in FIG. 7).

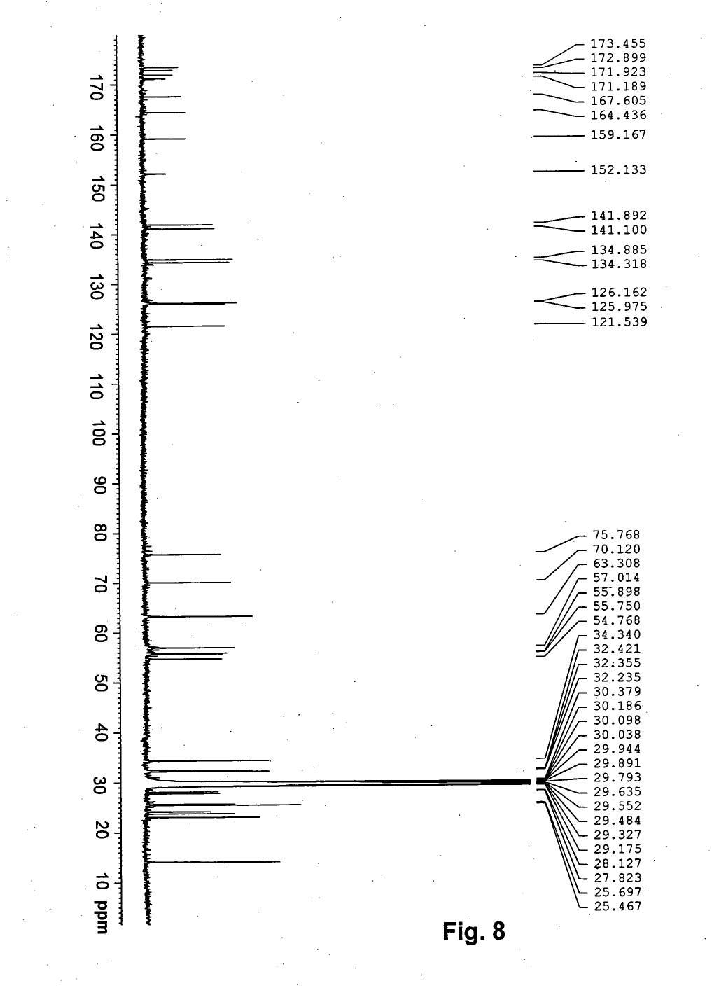

¹³C values (100 MHz, Acetone-d₆): 173.5 s, 172.9 s, 171.9 s, 171.1 s, 167.6 s, 164.5 s, 159.2 s, 152.2 s, 141.9 d, 141.1 d, 134.9 d, 134.3 d, 126.2 d, 126.0 d, 121.5 d, 75.8 d, 70.1 d, 63.3 t, 57.0 d, 55.9 d, 55.6 d, 54.8 d, 34.4 t, 32.4 t, 32.3 t, 29.3 t, 29.2 t, 28.2 t, 27.7 t, 25.7 q, 25.5 t, 24.2 q, 23.8 q, 23.1 t, 14.2 q. (¹³C NMR spectrum of Salarin A is showed in FIG. 8).

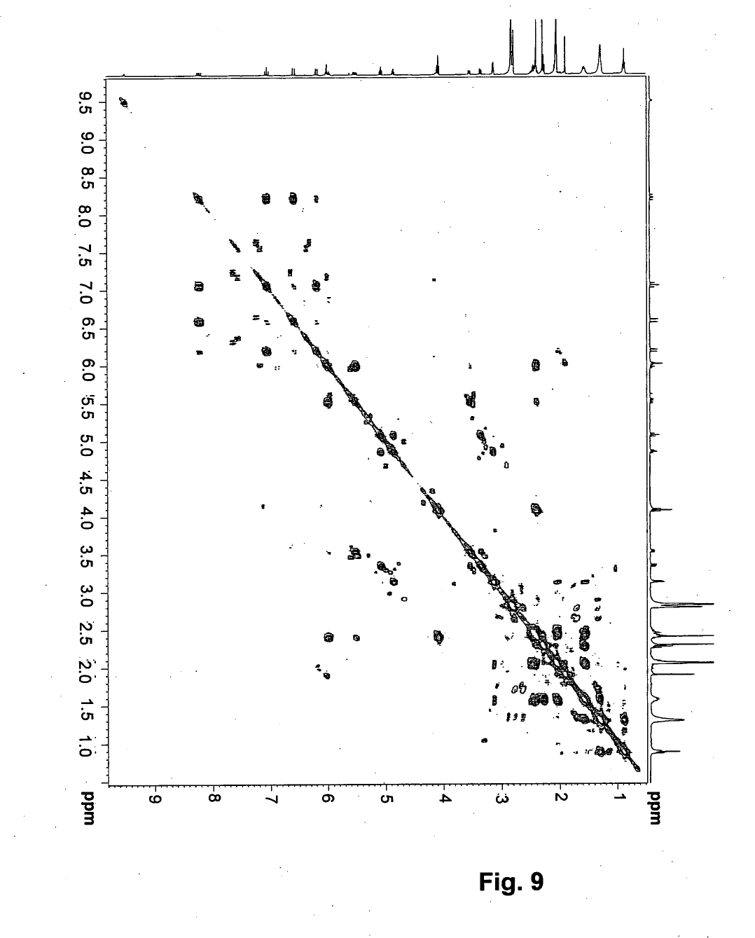

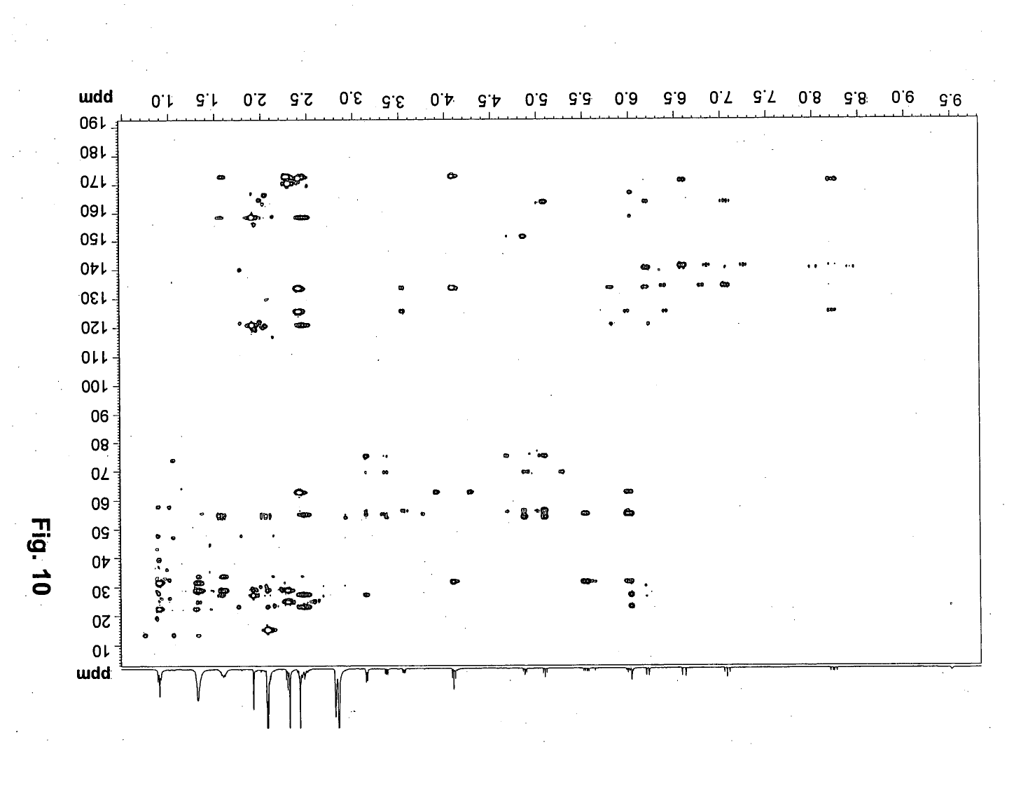

The ¹H, ¹³C, COSY (FIG. 9), HSQC and HMBC (FIG. 10) spectra (Table 1) revealed the presence of (a) two epoxides [δ 55.6 d and 54.8 d (E); δ 57.0 d and 55.9 d (Z)]; (b) two double bonds, (δ 126.2 d and 134.3 d as well as a conjugated one, δ 121.5 d and 159.2 s); (c) an octanoate ester (δ 173.5 s, 34.4 t, additional five methylenes and 14.2 q); (d) an 6-oxo-hexa-2,4-dienoate (δ 164.5 s, 126.0 d, 141.9 d, 141.1 d, 134.9 d and 171.9 s); (e) an N-acetyl carbamate (δ 152.2 s, 171.1 s and 24.2 q); and (f) a triacylamine (δ 171.9 s, 172.9 s, 25.7 q and 167.6 s). The exceptionally low field 8.21 signal, of H-4, agrees only with the 2Z, 4E isomer and requires a carbonyl at position C-6.

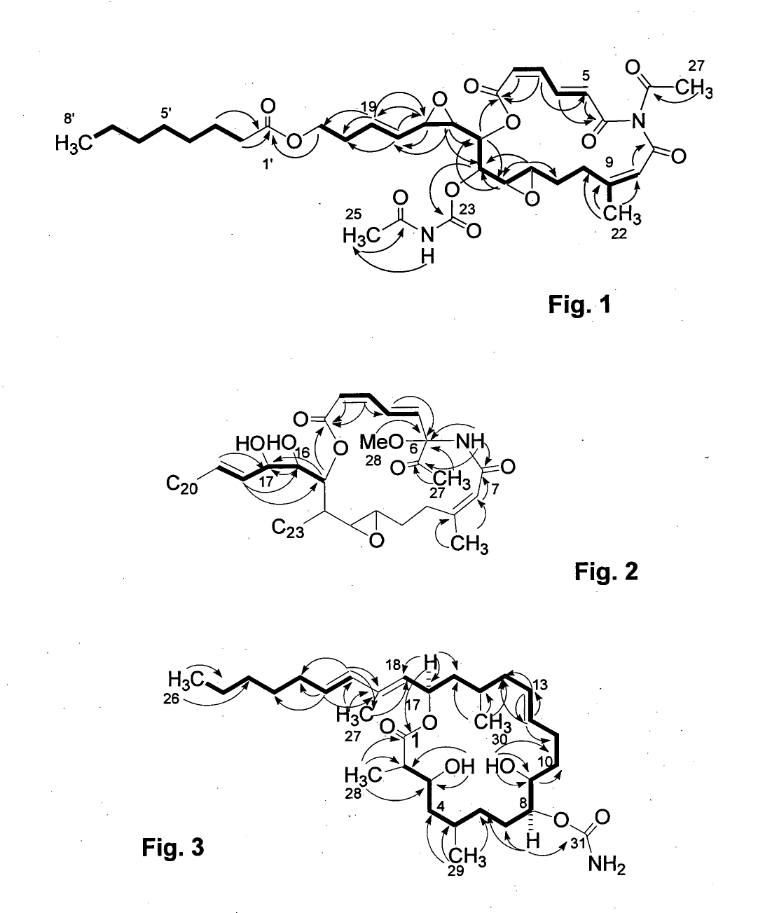

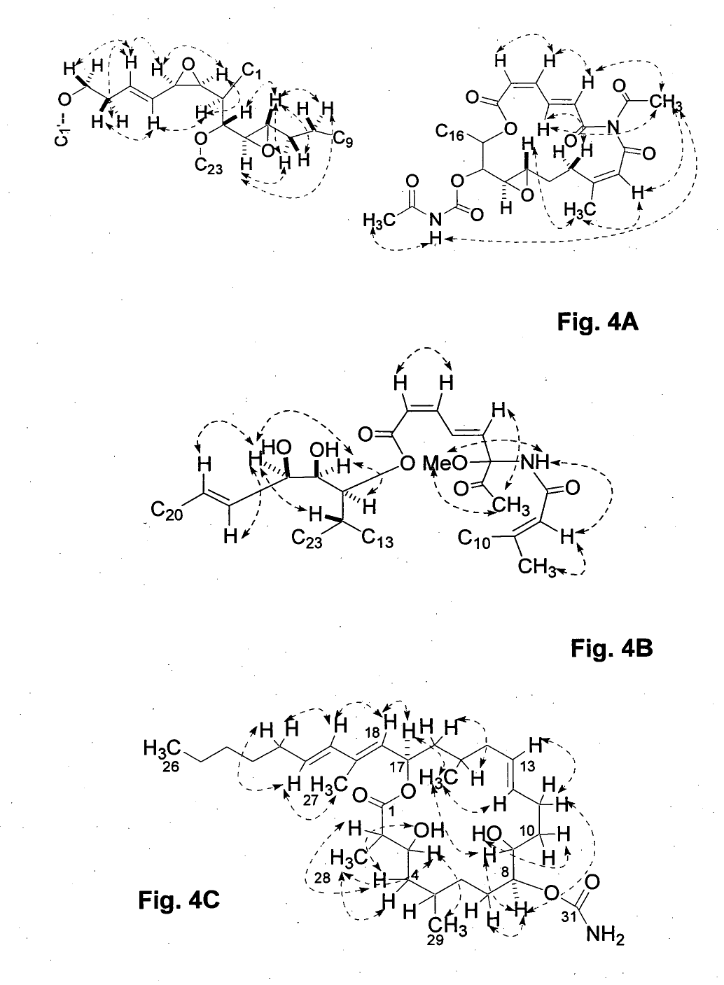

The naturally unique, N-acetyl carbamate group was suggested following CH- and NH-HMBC experiments (δ_{N} 143 ppm) and was in agreement with the acidity of the imide proton, among the two carbonyls, which could be methylated with CH₃D, in the presence of K₂CO₃ in acetone, to afford the N—CH₃ derivative (δ_{H} 3.23 s, δ_{C} 30.3 q). Assembling groups (a)-(e) via three unaccounted pairs of carbon atoms (C-10,11; 14,15 and 20,21), by COSY and HMBC data (Table 1, FIG. 1) afforded the structure of Salarin A lacking only a NCOCH₃ functionality (N—C26,27), proving as a result, the three acyl amine moiety (f). The stereochemistry of the double bonds and epoxides was determined by means of NOEs, consequentially completing the structure of Salarin A.

### Salarin B:

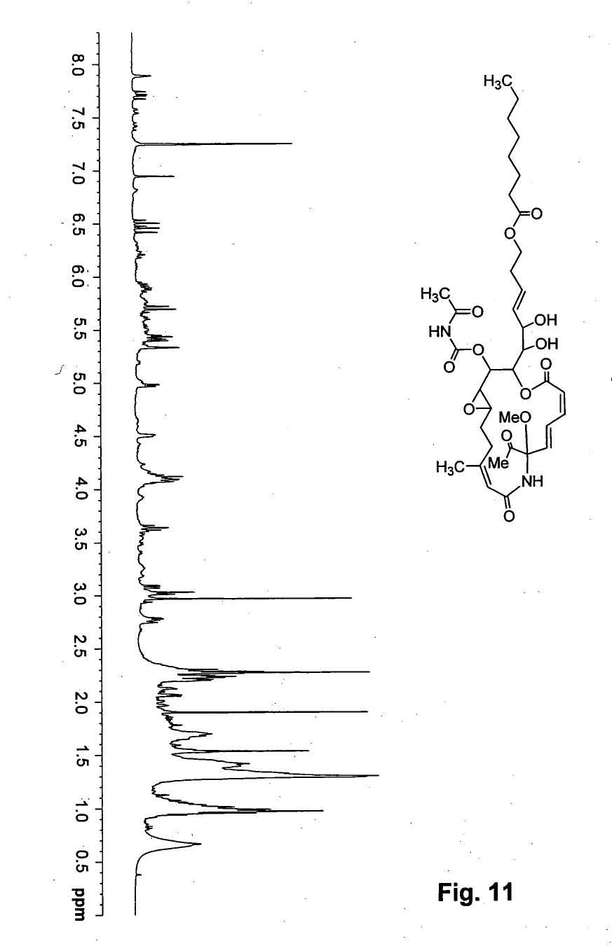

Colorless oil; [α]²³D −130 (c, 0.12, CHCl₃); HRESMS m/z 741.3028 [M+K]⁺ (Calcd. for C₃₆H₅₂N₂O₁₃K 741.2995), with 13 degrees of unsaturation. ¹H -δ values (500 MHz, Acetone-d₆): 7.89 s, 7.71 dd, 6.94 s, 6.50 t, 6.42 d, 5.89 dt, 5.71 d, 5.41 m, 5.40 m, 5.33 s, 4.98 dd, 4.51 m, 4.09 m, 3.63 t, 3.08 dd, 2.79 m, 2.28 m, 2.26 s, 2.22 m, 2.10 m, 1.96 m, 1.90 s, 1.68 m, 1.52 s, 1.41 m, 1.29 m. (¹H-NMR spectrum of Salarin B is showed in FIG. 11).

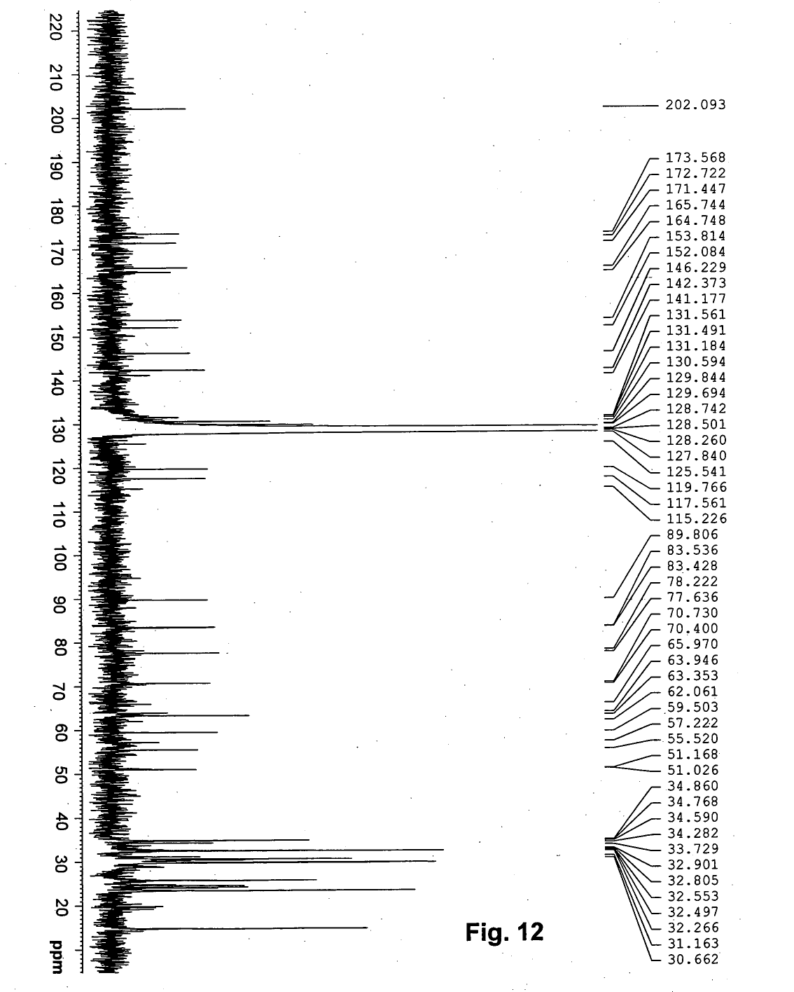

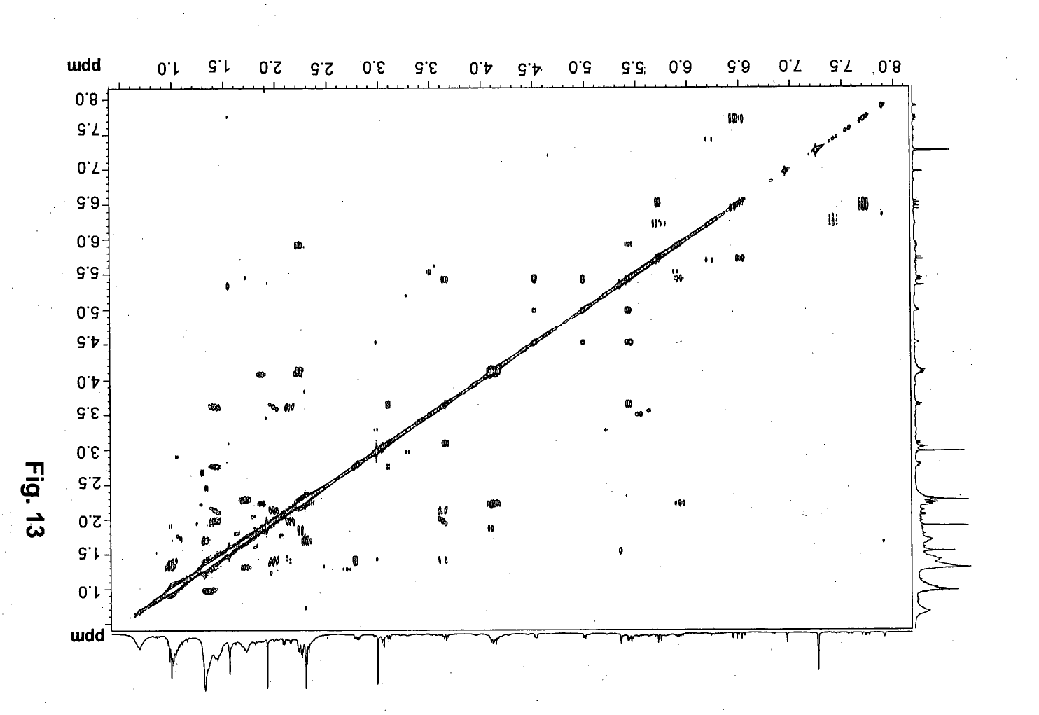

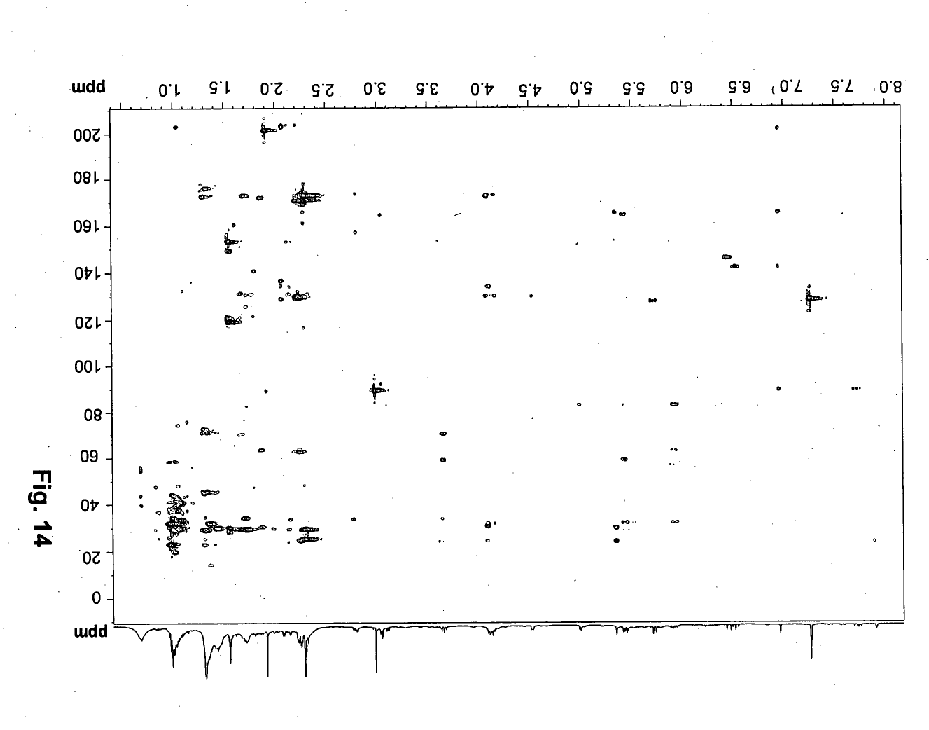

¹³C values-δ values (100 MHz, Acetone-d₆): 202.1 s, 173.6 s, 171.4 s, 165.7 s, 164.7 s, 153.8 s, 152.1 s, 146.2 d, 142.4 d, 131.5 d, 130.6 d, 127.8 d, 119.8 d, 117.5 d, 89.8 s, 83.4 d, 83.5 d, 77.6 d, 70.7 d, 63.3 t, 59.5 d, 55.5 d, 51.0 q, 34.8 t, 34.5 t, 32.5 t, 30.6 t, 29.9 t, 25.8 t, 24.6 q, 24.3 q, 24.0 q. (¹³C-NMR spectrum of Salarin B is showed in FIG. 12).

Salarin B lacks the 16,17-epoxide of Salarin A being replaced by a 16,17-diol and the triacylamine (f) is missing. Instead of the latter functionality, Salarin B possesses in the macrolide a lactam carrying next to the nitrogen atom (on C-6) a methoxyl and a methyl ketone (Table 2 and FIG. 2). The structure of the C₅-C₉ segment was suggested on the basis of 2D NMR data (FIGS. 2, 13 and 14). In addition, MS fragmentations of Salarin B confirmed conclusively the suggested structure, e.g. peaks of M-OCH₃, M-COCH₃ and m/z 257 for the C-15 side chain (C₁₄H₂₅O₄).

### Salarin C:

Bright orange oil; [α]²³D −64 (c 0.34, CHCl₃): IR(CHCl₃) νₘₐₓ 3648, 3054, 2986, 1717, 1421, 1272 cm⁻¹. UV (MeOH) affords three absorptions at 206, 270, 347 nm. The mass spectrometric analysis of Salarin C provided a molecular formula of C₃₅H₄₆N₂O₁₀Na HR-ESIMS (QqTOF) m/z 677.3035 for [M+Na]⁺ (calcd. for C₃₅H₄₆N₂O₁₀Na, 677.3044), with 14 degrees of unsaturation.

¹H-δ values (400 MHz, C₆D₆): 8.50 dd, 6.94 s, 6.47 t, 6.22 d, 5.88 m, 5.87 s, 5.75 dt, 5.72 d, 5.50 dd, 4.95 dd, 4.04 td, 3.75 dd, 3.72 m, 3.40 dd, 3.23 dt, 3.09 dd, 2.19 t, 2.14 m, 2.13 s, 2.00 m, 1.73 s, 1.59 m, 1.52 s, 1.51 m, 1.30 m, 1.22 m, 1.19 m, 0.88 t. (¹H-NMR spectrum of Salarin C is showed in FIG. 19).

¹³C values (100 MHz, C₆D₆): 172.7 s, 170.3 s, 164.9 s, 159.3 s, 151.2 s, 150.6 s, 145.6 s, 142.9 d, 133.7 d, 128.7 d, 128.7 s, 126.0 d, 124.6 d, 117.5 d, 110.6 d, 78.0 d, 67.9 d, 62.7 t, 56.8 d, 56.7 d, 56.1 d, 54.9 d, 34.0 t, 31.8 t, 31.7 t, 29.7 t, 29.5 t, 29.1 t, 29.0 t, 25.0 t, 24.6 q, 23.3 q, 22.7 t, 14.0 q, 9.3 q. (¹³C-NMR spectrum of Salarin C is showed in FIG. 20).

The ¹H, ¹³C (Table 4), COSY, HSQC, TOCSY, and HMBC spectra revealed the presence of the following moieties (a) two epoxides [δ 56.8 d and 54.9 d (E); δ 56.1 d and 56.7 d (Z)]; (b) an isolated double bond (δ 124.6 d and 133.7 d); (c) another double bond conjugated to a heterocycle (δ 110.6 d and 150.6 s); (d) an αβ, γδ-dienoate group (δ 164.9 s; 117.5 d and 142.9 d (Z); and δ 126.0 d and 128.7 d (E); (e) an octanoate ester (δ 172.7 s, 34.0 t, additional five methylenes and 14.0 q); (f) a 5-methyl three substituted oxazole (δ 128.7 s, 159.3 s, 145.6 s and 9.3 q) (g) an N-acetyl carbamate (δ 151.2 s, 170.3 s and 23.3 q).

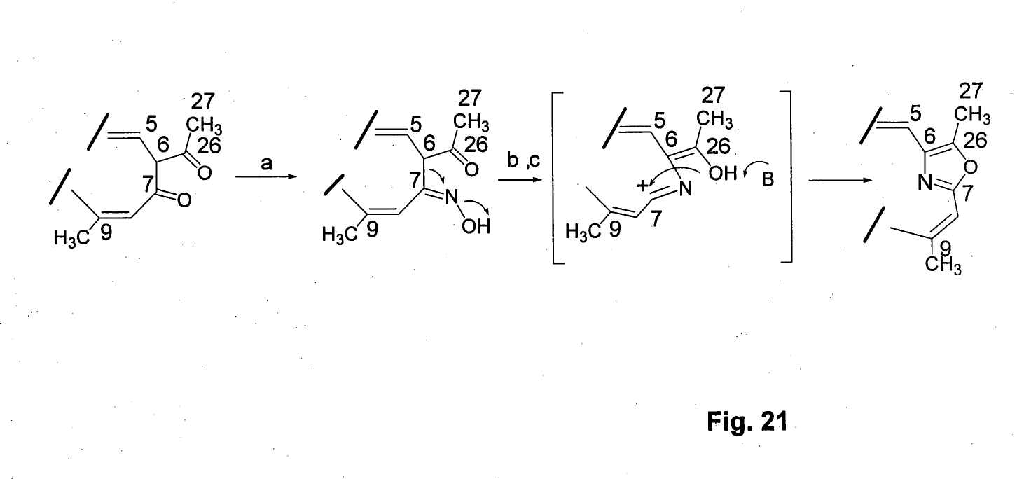

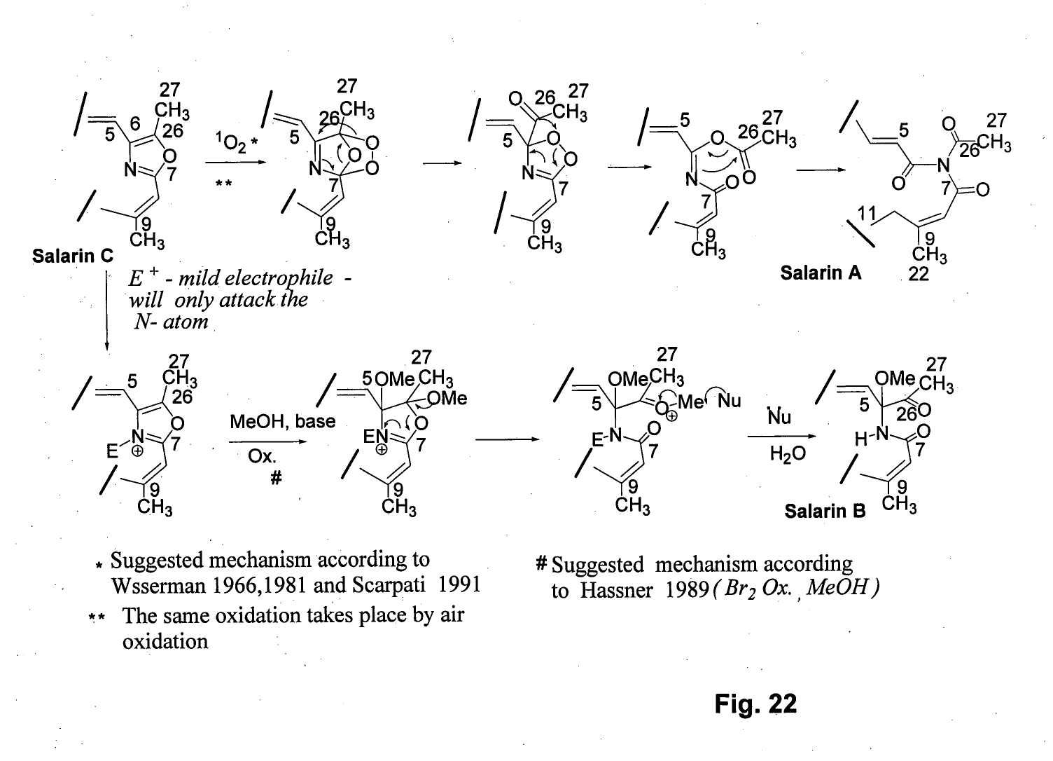

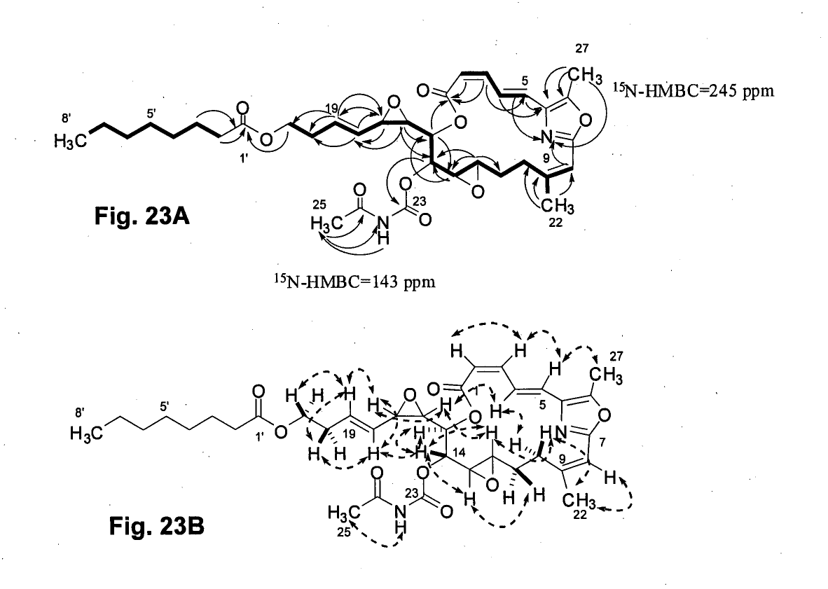

Strong support for the oxazole ring came from the ¹⁵N resonance, measured from the ³J(CH—N) HMBC correlation, of δ 245.0 ppm (in addition to the δ 143.0 ppm of the acetyl carbamate nitrogen atom). Assembling moieties a-g via three unaccounted for pairs of carbon atoms (C-10, 11 and 20, 21, methylenes, and C-14, 15 oxymethines) by COSY, HSQC, TOCSY and HMBC data (FIG. 21) afforded the gross structure of Salarin C. The Z, E, Z and E configuration of double bonds 2(3), 4(5), 8 (9) and 18 (19), respectively, and the E and Z configuration of the two epoxides, 12 (13) and 16 (17), respectively, of compound Salarin C were determined by the proper J values, measured NOEs and comparison to the suitable counterpart values in compounds Salarin A and Salarin B.

### Salarin D:

Yellow oil; [α]_{D}²⁶ −29 (c 0.6, CHCl₃). ¹H and ¹³C NMR data (C₆ D₆) of the signify region C14-C19; [δ_{C} 76.4 d (C-14), δ_{H} 5.09 t (3.6); δ_{C} 69.4 d (C-15), δ_{H} 5.50 dd (7.6, 3.6); δ_{C} 55.4 d (C-16), δ_{H} 3.44 m; δ_{C} 55.9 d (C-17), δ_{H} 3.38 brt (4.8); δ_{C} 124.5 d (C-18), δ_{H} 5.41 dd (15.7, 7.1); δ_{C} 133.6 d (C-19), δ_{H} 5.78 dt (15.7, 7.1)]. HR-ESIMS m/z 725.3205 [M+Na]⁺ (Calcd. For C₃₆H₅₀N₂O₁₂Na 725.3255).

### Salarin E:

Yellow oil; [α]_{D}²⁶ −98 (c 0.3, CHCl₃). ¹H and ¹³C NMR data (CDCl₃) for C1-C9; [δ_{C} 164.5 (C-1); δ_{C} 124.5 d, (C-2), δ_{H} 5.93 d (11.3); δ_{C} 140.4 d (C-3), δ_{H} 6.72 t (11.3); δ_{C} 137.9 d (C-4), δ_{H} 8.10 dd (16.1, 11.3); δ_{C} 131.6 d (C-5), δ_{H} 6.33 d (16.1); δ_{C} 166.9 s (C-6); δ_{C} 166.1 s (C-7); δ_{C} 120.3 d (C-8), δ_{H} 5.96 s; δ_{C} 150.7 s (C-9)]. FABMS m/z 667.0 [M+Na]⁺.

### Salarin F:

Yellow oil; [α]_{D}²⁶ −126 (c 0.13, CHCl₃). ¹H and ¹³C NMR data (CDCl₃) for C14-C19; [δ_{C} 77.0 d (C-14), δ_{H} 4.87 dd (9.1, 2.3); δ_{C} 71.4 d (C-15), δ_{H} 5.60 dd (8.7, 2.3); δ_{C} 73.2 d (C-16), δ_{H} 4.05 m; δ_{C} 65.3 d (C-17), δ_{H} 4.49 dd (7.4, 4.4); δ_{C} 128.7 d (C-18), δ_{H} 5.65 m; δ_{C} 131.4 d (C-19), δ_{H} 5.67 m]. FABMS m/z 655.0 [M−H₂O+H]⁺.

### Salarin G:

Yellow oil; [α]_{D}²⁶ −59 (c 0.29, CHCl₃). ¹H and ¹³C NMR data (CDCl₃) C14-C19; [δ_{C} 72.2 d (C-14), δ_{H} 5.20 t (4.4); δ_{C} 73.2 d (C-15), δ_{H} 5.38 dd (7.7, 4.4); δ_{C} 73.5 d (C-16), δ_{H} 3.99 t (7.7); δ_{C} 64.5 d (C-17), δ_{H} 4.57 dd (8.4, 4.5); δ_{C} 128.5 d (C-18), δ_{H} 5.71 dd (15.3, 7.2); δ_{C} 132.4 d (C-19), O_{H} 5.85 dt (15.3, 7.2]. FABMS m/z 727.1 [M+Na]⁺.

### Salarin C-1:

Colorless oil; [α]_{D}²³ 70 (c 0.2, CHCl₃). ¹H and ¹³C NMR data of the signify region C14-C19; [δ_{C} 76.4 d (C-14), δ_{H} 4.89 dd (8.6, 2.2); δ_{C} 72.9 d (C-15), δ_{H}5.48 dd (8.6, 2.2); δ_{C} 71.2 d (C-16), δ_{H} 4.45 m; δ_{C} 67.6 d (C-17), δ_{H} 4.15 m; δ_{C} 127.3 d (C-18), δ_{H} 5.67 dd (15.5, 6.1); δ_{C} 136.4 d (C-19), δ_{H} 5.74 m]. HR-ESIMS m/z 695.3135 [M+Na]⁺ (Calcd. For C₃₅H₄₈N₂O₁₁Na 695.3150).

### Salarin C-2:

¹H and ¹³C NMR data for C14-C30; [δ_{C} 76.4 d (C-14), δ_{H} 4.80 dd (8.7, 2.2); δ_{C} 72.3 d (C-15), δ_{H} 5.61 m; δ_{C} 78.3 d (C-16), δ_{H} 4.00 dd (9.8, 7.8); δ_{C} 82.4 d (C-17), δ_{H} 4.27 t (7.8); δ_{C} 127.5 (C-18), δ_{H} 5.41 dd (15.6, 8.1); δ_{C} 131.8 d (C-19), δ_{H} 5.64 m; (acetoneide moiety) δ_{C} 101.7 s (C-28); δ_{C} 28.7 q (two Me-29,30), δ_{H} 27.3 s. FABMS m/z 735.3 [M+Na]⁺.

### Salarin C-3:

¹H and ¹³C NMR data C1-C10; [δ_{C} 172.2 (C-1); δ_{C} 33.1 t (C-2); δ_{C} 25.7 t (C-3); δ_{C} 31.6 t (C-4); δ_{C} 23.9 (C-5); δ_{C} 133.2 s (C-6); δ_{C} 161.2 s (C-7); δ_{C} 35.4 t (C-8); δ_{C} 29.8 d (C-9); δ_{C} 30.6 t (C-10)]. NMR data C14-C20; [δ_{C} 76.3 (C-14), δ_{H} 4.77 dd; δ_{C} 75.8 (C-15), δ_{H} 5.34 m; δ_{C} 34.3 t (C-16); δ_{C} 71.1 d (C-17), δ_{H} 3.71 m; δ_{C} 35.1 t (C-18); δ_{C} 22.6 t (C-19); δ_{C} 28.5 t (C-20)]. FABMS m/z 687.3 [M+Na]⁺ (100); 665.3 [M+H]⁺ (35).

Due to proton signal overlap not all proton chemical shifts ¹H NMR for methylene protons were determined.

### Salarin C-4:

¹H and ¹³C NMR data of the signify region; [δ_{C} 76.1 d (C-14), δ_{H} 4.60 dd (8.9, 2.6); δ_{C} 157.8 s (C-23)]. There is no MS analyze suitable for formula C33H₄₄N₂O₉.

### Salarin A-1:

¹³C NMR data for C14-C19: [δ_{C} 73.1 d (C-14), δ_{H} 5.04 t (4.3); δ_{C} 73.4 d (C-15), δ_{H}5.44 dd (6.8, 4.3); δ_{C} 78.3 d (C-16), δ_{H} 3.94 t (6.8); δ_{C} 80.9 d (C-17), δ_{H} 4.37 t (8); δ_{C} 128.3 d (C-18), δ_{H} 5.48 dd (15.4, 7.9); δ_{C} 133.0 d (C-19), δ_{H} 5.77 dt (15.4, 7.9)]. There is no MS analyze suitable for formula C35H₄₈N₂O₁₃.

### Salain C-5:

¹H and ¹³C NMR data for substitute region: [δ_{C} 153.2 s (C-23); δ_{C} 171.4 s (C-24)]; p-bromobenzyl moiety: [δ_{C} 46.6 t (CH₂-28), δ_{H} 4.60 d (6.6); δ_{C} 153.4 s (C-29); δ_{C} 129.7 d (C-30,31), δ_{H} 7.11 d (8.2); δ_{C} 131.45 (C-32,33), δ_{H} 7.32 d (8.2); δ_{C} 121.2 s (C-34].

### Salarin A-2:

¹H and ¹³C NMR data for substitute region: [δ_{C} 153.8 s (C-23); δ_{C} 171.2 s (C-24)]; methyl moiety: [δ_{C} 30.3 q (C-28), δ_{H} 3.23 s]. HR-MALDIMS (TOF) m/z 723.3087 (Calcd. For C₃₆H₄₈N₂O₁₂Na 723.3099).

### Tulearin A:

Colorless oil; [α]²³_{D} −45 (c 0.17, CHCl₃); IR (CHCl₃) νₘₐₓ 3680 3430, 3020, 2960, 1729, 1602, 1582 cm⁻¹. ¹H and ¹³C NMR see Table 2. HRESMS m/z 558.3757 [M+Na]⁺ (Calcd. for C₃₁H₅₃NO₆Na, 558.3765), with six degrees of unsaturation.

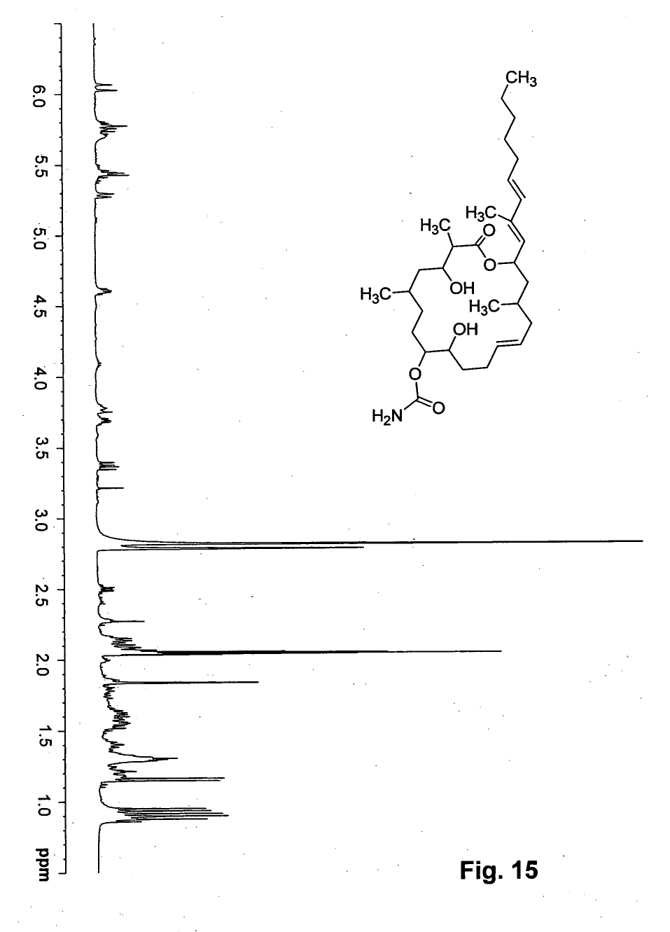

¹H-δ values (500 MHz, Acetone-d₆): 6.26 brs, 6.04 d, 5.80 dt, 5.75 dt, 5.44 m, 5.40 m, 5.28 d, 4.60 dt, 3.77 m, 3.69 dtd, 3.38 d, 3.35 d, 2.50 qd, 2.13 q, 2.08 q, 1.99 m, 1.85 s, 1.83 td, 1.78 m, 1.67 m, 1.62 m, 1.54 td, 1.51 m, 1.40 q, 1.32 m, 1.30 m, 1.29 m, 1.28 m, 1.24 td, 1.22 m, 1.17 brt, 1.15 d, 0.94 d, 0.90 d, 0.87 t. (¹H-NMR spectrum of Tulearin A is showed in FIG. 15).

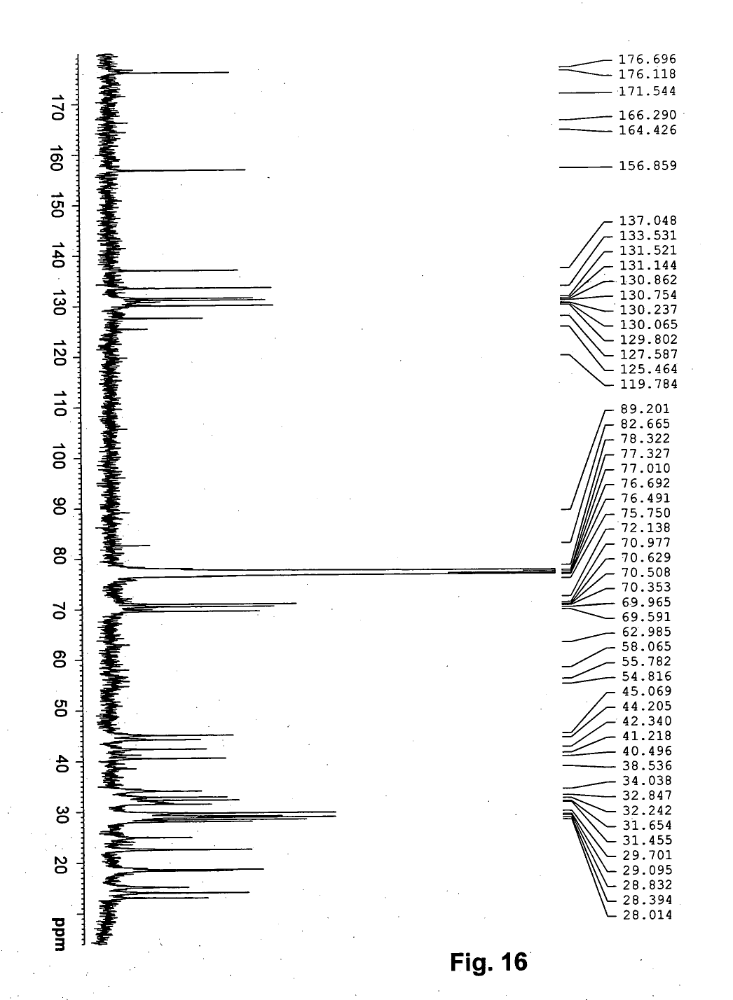

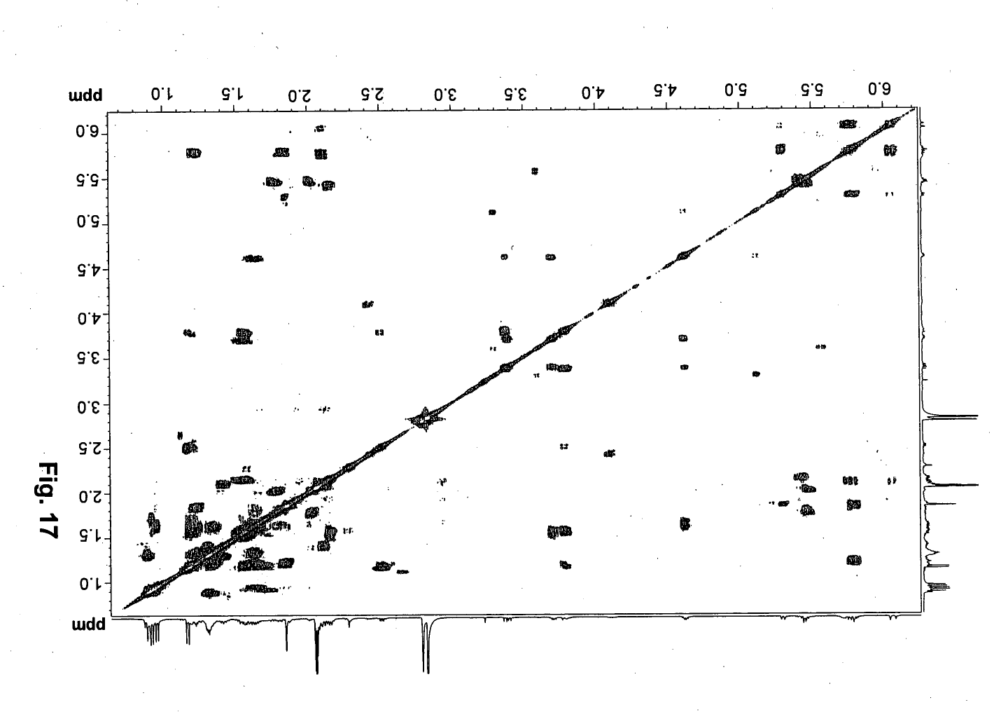

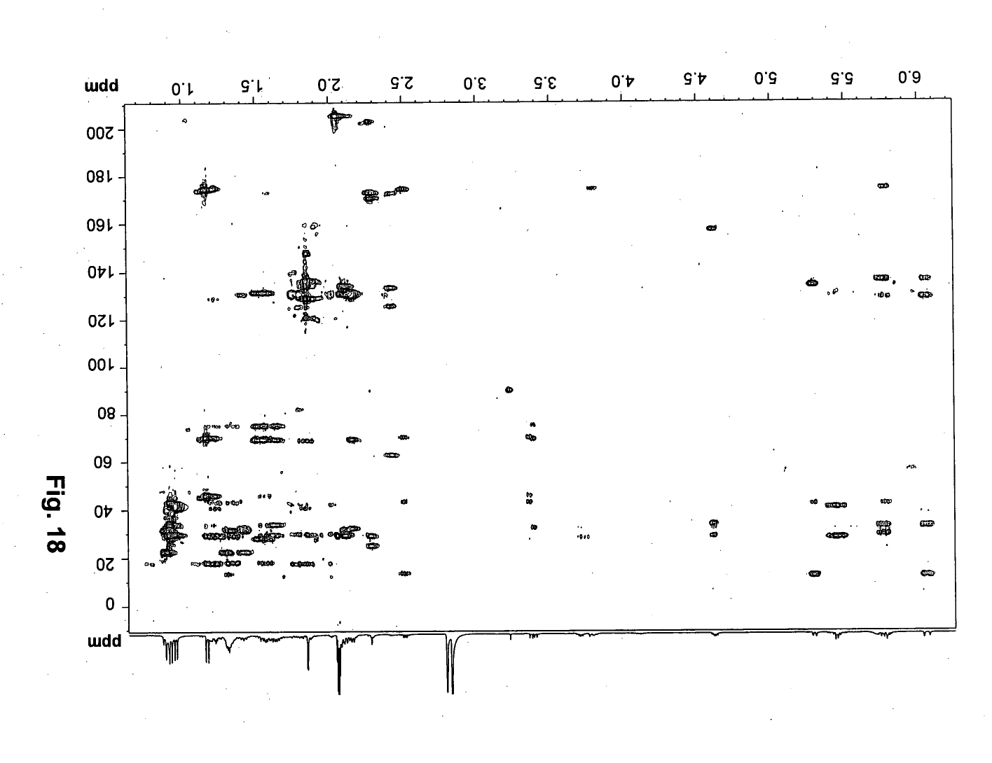

¹³C values (100 MHz, Acetone-d₆): 174.9 s, 157.7 s, 136.6 s, 134.6 d, 131.9 d, 131.2 d, 130.5 d, 129.5 d, 75.5 d, 70.8 d, 69.9 d, 69.6 d, 46.4 d, 43.8 t, 42.8 t, 41.3 t, 34.9 t, 33.6 t, 32.7 t, 31.3 t, 29.7 d, 29.4 t, 28.8 t, 28.0 d, 27.8 t, 23.0 t, 18.6 q, 18.2 q, 14.1 q, 14.0 q, 12.9 q. (¹³C-NMR spectrum of Tulearin A is showed in FIG. 16).

The ¹D and ²D NMR data (Table 3) revealed the presence of (a) an E,E-diene (δ 129.5 d, 136.6 s, 134.6 d and 131.2 d), and a non-conjugated E double bond (δ 131.9 d and 130.5 d); (b) two secondary alcohol groups (δ 70.8 d, 69.9 d); (c) a lactone (δ 174.9 s, 69.6 d); (d) five methyl groups (one triplet, one singlet, and three doublets (δ 14.1 q, 12.9 q, 14.0 q, 18.6 q, 18.2 q); and (e) a carbamate ester (δ 75.5 d, 157.7 s).

COSY and HMBC correlations (Table 3 and FIGS. 3, 17, 18) determined the complete planar structure of Tulearin A. Tulearin's A core is a 2,4,15,19-tetramethylated hexaeicosanoic, polyketide acid, possessing a 18 membered lactone (from C-1 to -17), carrying on the macrolide chain, besides two hydroxyls (on C-3 and 9), also a carbamate (on C-8).

### Tulearin B:

Yellow, amorphous powder; [α]_{D}²⁶ −37 (c 0.13, CHCl₃). ¹H and ¹³C NMR data (Actone-d₆) of the signify region C1-C4; [δ_{C} 172.1 (C-1); δ_{C} 43.9 d

### Tulearin A-1:

¹H and ¹³C NMR data for substitute region C17-C22: [δ_{C} 69.8 d (C-17), δ_{H} 5.62 dd; δ_{C} 58.1 d (C-18), δ_{H} 4.51 brs; δ_{C} 131.4 s (C-19); δ_{C} 123.9 d (C-20), δ_{H} 5.70 s; δ_{C} 56.7 d (C-21), δ_{H} 4.30 brs; δ_{C} 39.4 t (C-22), δ_{H} 1.74 m], δ_{C} 21.6 q (C-30), δ_{H} 1.91 s. TDPA substitute; [δ_{C} 153.4 s (C-32); δ_{C} 149.2 s (C-33); δ_{C} 129.3 s (C-34); δ_{C} 125.5 d (C-35,36), δ_{H} 7.48 d (8.4); δ_{C} 129.0 d (C-37,38), δ_{H} 7.44 d (8.4); δ_{C} 128.0 d (C-39), δ_{H} 7.34 t (7.1)]. FABMS m/z 711.0 [M+H]⁺ (80); 733.0 [M+Na]⁺ (100).

### Tulearin A-2:

¹H and ¹³C NMR data for substitute region C17-C22: [δ_{C} 69.4 d (C-17), δ_{H} 5.32 m; δ_{C} 57.3 d (C-18), δ_{H} 4.30 brs; δ_{C} 131.5 s (C-19); δ_{C} 123.5 d (C-20), δ_{H} 5.72 s; δ_{C} 56.5 d (C-21), δ_{H} 4.78 s; δ_{C} 36.6 t (C-22), δ_{H} 1.90 m, 123 m], δ_{C} 20.7 q (C-30), δ_{H} 1.95 s. TDPA substitute; [δ_{C} 154.3 s (C-32); δ_{C} 149.0 s (C-33); δ_{C} 128.2 s (C-34); δ_{C} 125.4 d (C-35,36), δ_{H} 7.54 d (8.4); δ_{C} 129.0 d (C-37,38), δ_{H} 7.60 d (8.4); δ_{C} 127.9 d (C-39), δ_{H} 7.32 t (7.1)]. FABMS m/z 711.0 [M+H]⁺ (15); 733.0 [M+Na]⁺ (100).

### Tulearin A-3:

¹H and ¹³C NMR data for substitute region C8-C10 [δ_{C} 72.1 d (C-8), δ_{H} 4.78 m; δ_{C} 81.5 d (C-9), δ_{H} 4.69 m; δ_{C} 31.3 t (C-10), δ_{H} 1.57 m]. Tosyl group (Ts): δ_{C} 127.5 s (C-32), δ_{C} 129.9 d (C-33,34), δ_{H} 7.35 d, δ_{C} 127.8 d (C-35,36), δ_{H} 7.79 d, δ_{C} 137.0 s (C-37), δ_{C} 21.0 q (C-38), δ_{H} 2.45 s.

### Tulearin A-4:

¹³C NMR data for substitute region C8-C10: [δ_{C} 74.3 d (C-8); δ_{C} 73.4 d (C-9); δ_{C} 31.4 t (C-10)]. Aryl group (Ar): δ_{C} 165.2 s (C-32), δ_{C} 137.0 s (C-33), δ_{C} 131.9 d (C-34,35), δ_{C} 131.4 d (C-36,37), δ_{C} 128.0 s (C-38).

### Tulearin A-5:

¹³C NMR data for substituted region C3 and C9: [δ_{C} 72.5 d (C-3); acetate group δ_{C} 170.5 s (CO); δ_{C} 21.3 q (CH₃); 77.1 d (C-9); acetate group δ_{C} 170.1 s (CO); δ_{C} 21.1 q (CH₃).

TABLE 1
NMR spectroscopic data for Salarin A.

| | δ_{C} | | δ_{H}, mult | ¹H-¹H | | HMBC |
|---|---|---|---|---|---|---|
| Position | (mult)^{a} | | (J in Hz)^{b} | COSY^{c} | NOESY | (H-C) |
| 1 | 164.5 | s | | | | |
| 2 | 126.0 | d | 6.18 d (11.3) | 3 | 3 | 1, 3 |
| 3 | 141.9 | d | 7.05 t (11.3) | 2, 4 | 2, 5 | 1, 4, 5 |
| 4 | 141.1 | d | 8.21 dd (15.7, 11.3) | 3, 5 | 10, 27 | 2, 3, 5, 6 |
| 5 | 134.9 | d | 6.58 d (15.7) | 4 | 3, 27 | 3, 4, 6 |
| 6 | 171.9 | s | | | | |
| 7 | 167.6 | s | | | | |
| 8 | 121.5 | d | 6.00 s | 22 | 22, 27 | 7, 9, 10, 22 |
| 9 | 159.2 | s | | | | |
| 10 | 27.7 | t | 2.48 m (2H) | 11a, 11b | 4, 12 | 8, 9, 11, 12, 22 |
| 11 | 28.2 | t | 2.04 m | 10, 11b, 12 | 12 | 10, 12, 13 |
| | | | 1.55 m | 10, 11a, 12 | 12 | 9, 10, 12, 13 |
| 12 | 55.6 | d | 3.12 m^{d} | 11a, 11b, 13 | 11a, 11b, 14, | 11, 13, 14, 15 |
| | | | | | 22 | |
| 13 | 54.8 | d | 3.13 m^{c} | 12, 14 | 10, 11b, 14 | 12, 14, 15 |
| 14 | 75.8 | d | 4.84 t (7.6) | 13, 15 | 12, 13, 15, 16 | 13, 15, 16, 23 |
| 15 | 70.1 | d | 5.06 t (7.6) | 14, 16 | 13, 14, 16, 18 | 1, 13, 14, 16, 17 |
| 16 | 57.0 | d | 3.34 dd (7.6, 4.0) | 15, 17 | 14, 15, 17 | 14, 15, 17 |
| 17 | 55.9 | d | 3.53 dd (6.8, 4.0) | 16, 18 | 16, 18, 19 | 16, 18, 19 |
| 18 | 126.2 | d | 5.52 dd (15.6, 6.8) | 17, 19 | 15, 17, 20 | 17, 20 |
| 19 | 134.3 | d | 5.98 dt (15.6, 6.8) | 18, 20 | 17, 20, 21 | 17, 20, 21 |
| 20 | 32.4 | t | 2.37 q (6.8) (2H) | 19, 21 | 18, 19, 21 | 18, 19, 21 |
| 21 | 63.3 | t | 4.08 t (6.8) (2H) | 20 | 19, 20 | 19, 20, 1′ |
| 22 | 23.8 | q | 1.90 s | 8 | 8 | 8, 9, 10 |
| 23 | 152.2 | s | | | | |
| NH | | | 9.48 s | | 25, 27 | 25 |
| 24 | 171.1 | s | | | | |
| 25 | 24.2 | q | 2.30 s | | NH | 24 |
| 26 | 172.9 | s | | | | |
| 27 | 25.7 | q | 2.39 s | | 4, NH | 26 |
| 1′ | 173.5 | s | | | | |
| 2′ | 34.4 | t | 2.28 t (7.2) (2H) | 3′ | 3′, 4′ | 1′, 3′, 4′ |
| 3′ | 25.5 | t | 1.58 m (2H) | 4′ | | 2′, 4′ |
| 4′ | 29.2 | t | 1.30 m (2H) | 3′, 5′ | | 3′, 5′ |
| 5′ | 29.3 | t | 1.30 m (2H) | 4′, 6′ | | 4′, 6′ |
| 6′ | 32.3 | t | 1.28 m (2H) | 5′, 7′ | | 5′, 7′ |
| 7′ | 23.1 | t | 1.29 m (2H) | 6′, 8′ | | 5′, 6′, 8′ |
| 8′ | 14.2 | q | 0.87 t (6.9) | 6′, 7′ | | 6′, 7′ |

^{a}Data recorded in Acetone-d₆ on Bruker Avance 500 and 100 MHz instrument (100 MHz for ¹³C).
^{b}The CH correlation were assigned by an HSQC experiment.
^{c}a, b geminal pair denote, the upper (a) and lower (b) protons.
^{d}Protons 12 and 13 were well separated in C₆D₆; H-12 δ_{H} 2.89 dd, 7.1, 2.2 Hz and H-13 δ_{H} 3.15 dd, 4.2, 2.2 Hz.

TABLE 2
NMR spectroscopic data for Salarin B.

| | δ_{C} | | δ_{H}, mult | ¹H-¹H | | HMBC |
|---|---|---|---|---|---|---|
| Position | (mult)^{a} | | (J in Hz)^{b} | COSY^{c} | NOESY | (H-C) |
| 1 | 164.7 | s | | | | |
| 2 | 117.5 | d | 5.71 d (11.2) | 3 | 3 | 1, 3 |
| 3 | 146.2 | d | 6.50 t (11.2) | 2, 4 | 2 | 1, 4, 5 |
| 4 | 127.8 | d | 7.71 dd (15.4, 11.2) | 3, 5 | 27 | 2, 3, 5, 6 |
| 5 | 142.4 | d | 6.42 d (15.4) | 4 | 27 | 3, 4, 6 |
| 6 | 89.8 | s | | | | |
| NH | | | 6.94 s | | 8, 28 | 5, 6, 7, 26 |
| 7 | 165.7 | s | | | | |
| 8 | 119.8 | d | 5.33 s | 22 | NH, 22 | 7, 9, 10, 22 |
| 9 | 153.8 | s | | | | |
| 10 | 30.6 | t | 2.10 m (2H) | 11a, 11b | 12 | 8, 9, 11, 12, 22 |
| 11 | 34.5 | t | 1.96 m | 10, 11b, 12 | 12 | 10, 12, 13 |
| | | | 1.41 m | 10, 11a, 12 | 12 | 9, 10, 12, 13 |
| 12 | 55.5 | d | 2.79 m | 11a, 11b, 13 | 14 | 11, 13, 14, 15 |
| 13 | 59.5 | d | 3.08 dd (8.0, 1.9) | 12, 14 | 11, 15 | 12, 14, 15 |
| 14 | 83.4 | d | 3.63 t (8.0) | 13, 15 | 12, 17 | 13, 15, 23 |
| 15 | 70.7 | d | 5.40 m^{d} | 14, 16 | 13, 16 | 1, 13, 14, 16 |
| 16 | 77.6 | d | 4.98 dd (6.4, 2.9) | 15, 17 | 15 | 14, 15, 17 |
| 17 | 83.5 | d | 4.51 m | 16, 18 | 14, 18, 19 | 16, 18, 19 |
| 18 | 130.6 | d | 5.41 m^{d} | 17, 19 | 17, 20 | 17, 20 |
| 19 | 131.5 | d | 5.89 dt (14.9, 6.8) | 18, 20 | 17, 20, 21 | 17, 20, 21 |
| 20 | 32.5 | t | 2.22 m (2H)^{d} | 19, 21 | 18, 19, 21 | 18, 19, 21 |
| 21 | 63.3 | t | 4.09 m (2H)^{d} | 20 | 19, 20 | 19, 20, 1′ |
| 22 | 24.6 | q | 1.52 s | 8 | 8 | 8, 9, 10 |
| 23 | 152.1 | s | | | | |
| NH′ | | | 7.89 s | | 28 | 25 |
| 24 | 171.4 | s | | | | |
| 25 | 24.3 | q | 2.26 s | | | 24 |
| 26 | 202.1 | s | | | | 27 |
| 27 | 24.0 | q | 1.90 s | | 4, 5, 28 | 6, 26 |
| 28 | 51.0 | q | | | 27, NH, | 6 |
| | | | | | NH′ | |
| 1′ | 173.6 | s | | | | |
| 2′ | 34.8 | t | 2.28 m (2H)^{d} | 3′ | | 1′, 3′ |
| 3′ | 25.8 | t | 1.68 m (2H)^{d} | 4′ | | 2′, 4′ |
| 4′ | 29.9 | t | 1.29 m (2H)^{d} | 3′, 5′ | | 3′, 5′ |
| 5′ | 29.8 | t | 1.29 m (2H)^{d} | 4′, 6′ | | 4′, 6′ |
| 6′ | 32.4 | t | 1.28 m (2H)^{d} | 5′, 7′ | | 5′, 7′ |
| 7′ | 23.4 | t | 1.33 m (2H)^{d} | 6′, 8′ | | 5′, 6′, 8′ |
| 8′ | 14.2 | q | 0.97 t (6.8) | 6′, 7′ | | 6′, 7′ |

^{a}Data recorded in Acetone-d₆ on Bruker Avance 500 and 100 MHz instrument (100 MHz for ¹³C).
^{b}The CH correlation were assigned by an HSQC experiment.
^{c}a, b for a geminal pair denote the upper (a) and lower (b) protons.
^{d}Multiplicities were not determined because of overlapping with other signals.

TABLE 3
NMR spectroscopic data for Tulearin A.

| | δ_{C} | | δ_{H}, mult | ¹H-¹H | | HMBC |
|---|---|---|---|---|---|---|
| Position | (mult)^{a} | | (J in Hz)^{b} | COSY^{c} | NOESY | (H-C) |
| 1 | 174.9 | s | | | | |
| 2 | 46.4 | d | 2.50 qd (7.6, 2.8) | 3, OH | 3, 4b, 28 | 1, 3, 4, 28 |
| 3 | 70.8 | d | 3.77 m | 2, 4a, 4b, OH | 2, 28, 29, OH | 2, 5, 28 |
| OH | | | 3.35 d (7.6) | 2, 3 | 3, 4b | 2, 3, 4 |
| 4 | 43.8 | t | 1.54 td (13.7, 4.4) | 3, 5 | 5, 4b, 28 | 2, 3, 5, 29 |
| | | | 1.17 br t (13.7) | | 2, 4a, OH | 2, 3 |
| 5 | 28.0 | d | 1.67 m^{d} | 4b, 6b, 29 | 4a, 29 | 6, 29 |
| 6 | 34.9 | t | 1.32 m^{d} | 6b, 7 | 8, 9 | 4, 5, 7, 29 |
| | | | 1.22 m^{d} | 5, 6a, 7 | 6b, 7, 29 | 4, 5, 7, 29 |
| 7 | 28.8 | t | 1.62 m (2H)^{c} | 6a, 6b, 8 | 6b, 8, 29 | 4, 5, 8, 9 |
| 8 | 75.5 | d | 4.60 td (5.5, 3.9) | 7, 9, OH | 6a, 7, 9, 11 | 6, 7, 31 |
| 9 | 69.9 | d | 3.69 dtd (7.2, 6.7, 3.9) | 8, 10, OH | 6a, 8, 10, 12, 30, | 10 |
| | | | | | OH | |
| OH | | | 3.38 d (7.2) | 8, 9 | 9, 10 | 9, 10 |
| 10 | 32.7 | t | 1.51 m (2H)^{d} | 9, 11 | 9, 11, OH | 8, 9, 11, 12 |
| 11 | 27.8 | t | 2.13 q (6.0) (2H) | 10, 12 | 8, 10, 13 | 9, 10, 12, 13 |
| 12 | 131.9 | d | 5.40 m^{d} | 11 | 10, 15, 30 | 11, 13, 14 |
| 13 | 130.5 | d | 5.44 m^{d} | 14a, 14b | 11, 14a, 14b | 12, 14 |
| 14 | 41.3 | t | 1.99 m^{d} | 13, 14b | 13, 14b, 15 | 13, 15, 16, 30 |
| | | | 1.78 m^{d} | 13, 14a, 15 | 13, 14a, 30 | 15, 30 |
| 15 | 29.7 | d | 1.62 m^{d} | 14b, 16b, 30 | 12, 16b, 14a | 17, 30 |
| 16 | 42.8 | t | 1.83 td (13.1, 4.4) | 16b, 17 | 16b, 15, 18 | 14, 15, 17, 30 |
| | | | 1.24 td (13.1, 4.4) | 15, 16a, 17 | 16a, 17 | 15 |
| 17 | 69.6 | d | 5.80 td (9.1, 4.4) | 16a, 16b, 18 | 16b, 18, 27, 30 | 15, 18, 19 |
| 18 | 129.5 | d | 5.28 d (9.1) | 17, 27 | 16a, 20 | 20, 27 |
| 19 | 136.6 | s | | | | |
| 20 | 134.6 | d | 6.04 d (15.5) | 21, 22 | 18, 22 | 18, 19, 22, 27 |
| 21 | 131.2 | d | 5.75 dt (15.5, 6.9) | 20, 22 | 22, 27 | 19, 22, 23 |
| 22 | 33.6 | t | 2.08 q (7.0) (2H) | 21, 23 | 20, 21, 23a | 20, 21, 23, 24 |
| 23 | 29.4 | t | 1.40 quin (7.0) | 22, 23b | 22 | 22, 24, 25 |
| | | | 1.29 m^{d} | 23a | | 25 |
| 24 | 31.3 | t | 1.28 m (2H)^{d} | 23a, 25 | | 23, 25, 26 |
| 25 | 23.0 | t | 1.30 m (2H)^{d} | 24, 26 | | 24, 26 |
| 26 | 14.1 | q | 0.87 t (7.0) | 24 | | 24, 25 |
| 27 | 12.9 | q | 1.85 s | 18 | 17, 21 | 18, 19, 20 |
| 28 | 14.0 | q | 1.15 d (6.5) | 2 | 2, 3, 4a | 1, 2, 3 |
| 29 | 18.6 | q | 0.90 d (7.1) | 5 | 3, 5, 6b, 7 | 4, 5, 6 |
| 30 | 18.2 | q | 0.94 d (6.7) | 15 | 9, 12, 14b | 14, 15, 16 |
| 31 | 157.7 | s | | | | |

| | |
|---|---|
| NH₂ | 6.26 br s^{e} |

^{a}Data recorded in Acetone-d₆ on Bruker Avance 500 and 100 MHz instrument (100 MHz for ¹³C).
^{b}The CH correlation were assigned by an HSQC experiment.
^{c}a, b for a geminal pair denote the upper (a) and lower (b) protons.
^{d}Multiplicities were not determined because of overlapping with other signals.
^{e}The shift of NH₂ is given in DMSO-d₆ spectra.

TABLE 4
NMR spectroscopic data for Salarin C.

| | δ_{C} | | δ_{H}, mult | ¹H-¹H | | HMBC |
|---|---|---|---|---|---|---|
| Position | (mult)^{a} | | (J in Hz)^{b} | COSY^{c} | NOESY | (H-C) |
| 1 | 164.9 | s | | | | |
| 2 | 117.5 | d | 5.72 d (12.1) | 3 | 3 | 1, 3 |
| 3 | 142.9 | d | 6.47 t (12.1) | 2, 4 | 2, 5 | 1, 4, 5 |
| 4 | 126.0 | d | 8.50 dd (15.0, 12.1) | 3, 5 | 3, 10a, 16 | 2, 3, 5, 6 |
| 5 | 128.7 | d | 6.22 d (15.0) | 4 | 3, 27 | 3, 4, 6, 26 |
| 6 | 128.7 | s | | | | |
| 7 | 159.3 | s | | | | |
| 8 | 110.6 | d | 5.87 s | 22 | 22 | 7, 9, 10, 22 |
| 9 | 150.6 | s | | | | |
| 10 | 29.7 | t | 3.72 m^{d} | 10b, 11a, 11b | 4, 10b, 11a, | 8, 9, 11, 12, 22 |
| | | | 2.00 m | 10a, 11a | 22 | |
| | | | | | 10a, 12 | |
| 11 | 29.5 | t | 1.51 m | 10, 11b, 12 | 10a, 10b, 12 | 10, 12, 13 |
| | | | 1.30 m | 10, 11a, 12 | 11a | 9, 10, 12, 13 |
| 12 | 56.8 | d | 3.23 dt (9.0, 2.2) | 11a, 11b, 13 | 10a 10b, | 11, 13, 14, 15 |
| | | | | | 11a, 14, 16 | |
| 13 | 54.9 | d | 3.75 dd (9.0, 2.2) | 12, 14 | 10b, 11a, | 12, 14, 15 |
| | | | | | 11b, 14 | |
| 14 | 78.0 | d | 4.95 dd (9.0, 2.9) | 13, 15 | 12, 13, 15 | 13, 15, 16, 23 |
| 15 | 67.9 | d | 5.88 m^{d} | 14, 16 | 14, 18 | 1, 13, 14, 16, 17 |
| 16 | 56.1 | d | 3.40 dd (7.8, 3.9) | 15, 17 | 12, 17 | 14, 15, 17 |
| 17 | 56.7 | d | 3.09 dd (6.4, 3.9) | 16, 18 | 16, 18, 19 | 16, 18, 19 |
| 18 | 124.6 | d | 5.50 dd (15.3, 6.4) | 17, 19 | 15, 17, 20 | 17, 20 |
| 19 | 133.7 | d | 5.75 dt (15.3, 6.4) | 18, 20 | 17, 20, 21 | 17, 20, 21 |
| 20 | 31.8 | t | 2.14 m^{d} (2H) | 19, 21 | 18, 19, 21 | 18, 19, 21 |
| 21 | 62.7 | t | 4.04 td (6.9, 2.9) (2H) | 20 | 19, 20 | 19, 20, 1′ |
| 22 | 24.6 | q | 1.52 s | 8 | 8, 10b | 8, 9, 10 |
| 23 | 151.2 | s | | | | |
| NH | | | 6.94 s | | 25 | 25 |
| 24 | 170.3 | s | | | | |
| 25 | 23.3 | q | 2.13 s | | NH | 24 |
| 26 | 145.6 | s | | | | |
| 27 | 9.3 | q | 1.73 s | | 5 | 6, 26 |
| 1′ | 172.7 | s | | | | |
| 2′ | 34.0 | t | 2.19 t (7.8) (2H) | 3′ | 3′, 4′ | 1′, 3′, 4′ |
| 3′ | 25.0 | t | 1.59 m (2H) | 4′ | | 2′, 4′ |
| 4′ | 29.0 | t | 1.22 m (2H) | 3′, 5′ | | 3′, 5′ |
| 5′ | 29.1 | t | 1.22 m (2H) | 4′, 6′ | | 4′, 6′ |
| 6′ | 31.7 | t | 1.19 m (2H) | 5′, 7′ | | 5′, 7′ |
| 7′ | 22.7 | t | 1.22 m (2H) | 6′, 8′ | | 5′, 6′, 8′ |
| 8′ | 14.0 | q | 0.88 t (6.8) | 6′, 7′ | | 6′, 7′ |

^{a}Data recorded in C6D6-d₆ on Bruker Avance 400 MHz instruments (100 MHz for ¹³C).
^{b}The CH correlation were assigned by an HSQC experiment.
^{c}a, b, a geminal pair, denote the upper (a) and lower (b) protons. Multiplicities were not determined because of overlapping with other signals.

### E. Bio-Activity of the Various Compounds

The anti-cancer activities of the compounds of the invention have been determined in-vitro in human and mouse leukemia cell lines. The procedure was carried out using the colorimetric methyl thiazol tetrazoliumbromide (MTT) assay for measuring cell proliferation (Carmichael et al., (1987), Cancer Res. 47: 936-942).

Cell Culture

The Human leukemia cell lines K562 (Lozzio and Lozzio (1975) Blood, 45, 321-334) and UT7 (Komatsu et al. (1991), Cancer Res. 51, 341-348) were grown in RPMI 1640 and IMDM medium respectively, supplemented with 10% fetal bovine serum (FBS), 2 mM L-glutamine, 100 ug/ml streptomycin and 100 u/ml penicillin. The murine Ba/F3 cell line stably transfected with the erythropoietin receptor (EPO-R) cDNA (D'Andrea et al., (1991), Mol. Cell. Biol. 11, 1980-1987) was cultured in RPMI medium supplemented with recombinant human EPO (rHuEPO, 0.5 U/ml). UT7 cells were cultured in the presence of 2 U/ml rHuEPO. Cells were maintained at 37° C. in a 5% CO₂ humidified incubator.

Colorimetric MTT Assay

Cells (4×10³) were seeded in triplicate into 96-well, flat-bottom culture plates and grown in the presence of the compounds at different concentrations for 24, 48 and 72 hours. Untreated cells served as control. After incubation with the compound, cell growth was determined using the colorimetric methylthiazole tetrazolium bromide (MTT) assay (Mosmann (1983), J Immunol Methods 65, 55-63). Briefly, MTT was added to a final concentration of 5 μg/ml to each well and further incubated for 4 hours at 37° C. After complete solubilization of the dye by acid/alcohol (0.04 N HCl in 2-propanol), plates were read at 570 nm in an ELISA reader, reference 690 nm. Growth of cells exposed to treatment was calculated as the percent of optical density (OD) of the compound-treated cells to that of the non-treated cells.

The potential effects of Salarin A on cell proliferation was tested on two different human leukemia cell lines K562 and UT7 used as targets and treated with various concentrations of Salarin A for 24, 48 and 72 hours.

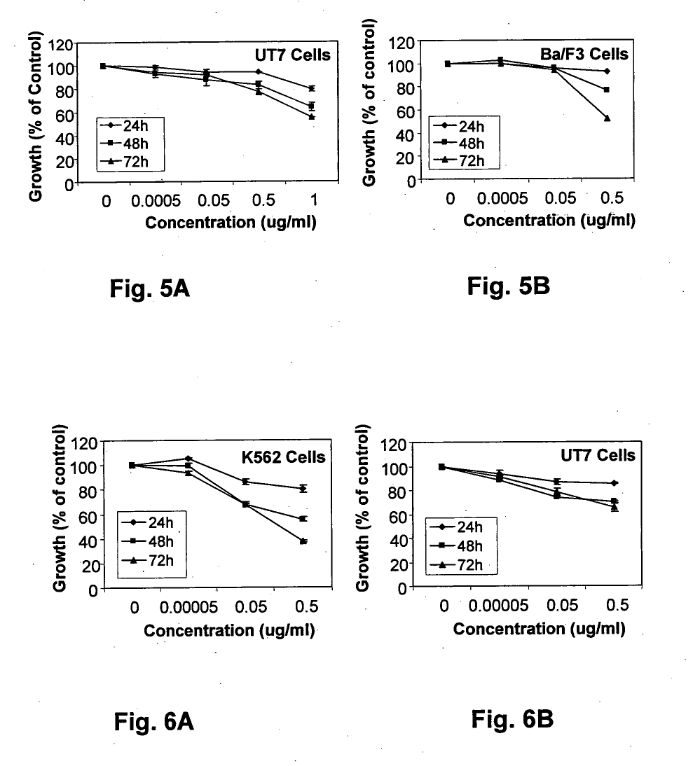

Salarin A induced inhibition of cell growth in EPO-dependent UT7 cells (FIG. 5), in a dose and time dependent manner. A concentration of 1 μg/ml of Salarin A induced 45% inhibition of cell proliferation in UT7 cells, after 72 h of exposure to the product (FIG. 5A). Salarin A was also tested in the murine EPO-dependent Ba/F3 cells expressing the EPO-R. In these cells 0.5 ug/ml of Salarin A induced 48% inhibition of cell proliferation after 72 h of exposure to the product (FIG. 5B).

To investigate the potential effects of Tulearin A on cell proliferation, two different human leukemia cell lines K562 and UT7 were used as targets and treated with various concentrations of 3 for 24, 48 and 72 hours. As shown in FIG. 6, Tulearin A induced inhibition of cell growth in K562 (A) and UT7 (B), in a dose and time dependent manner. K562 cells displayed a greater sensitivity to Tulearin A, as compared to UT7. Namely, a concentration of 0.5 ug/ml of Tulearin A induced 62% inhibition of cell proliferation in K562 cells (A) and 35% in UT7 cells (B), after 72 h of exposure to the compound.

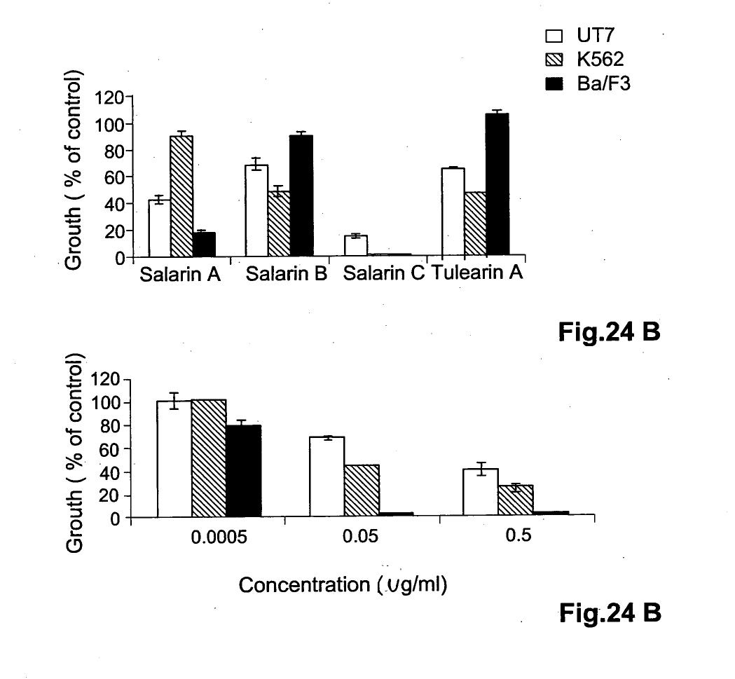

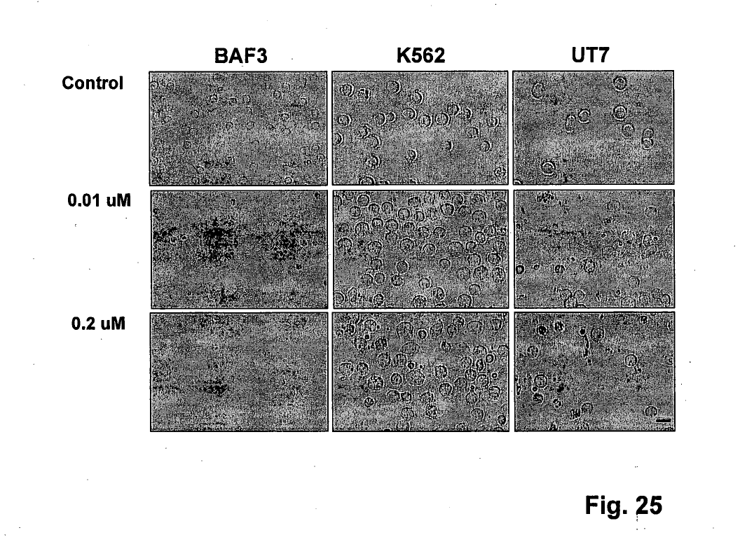

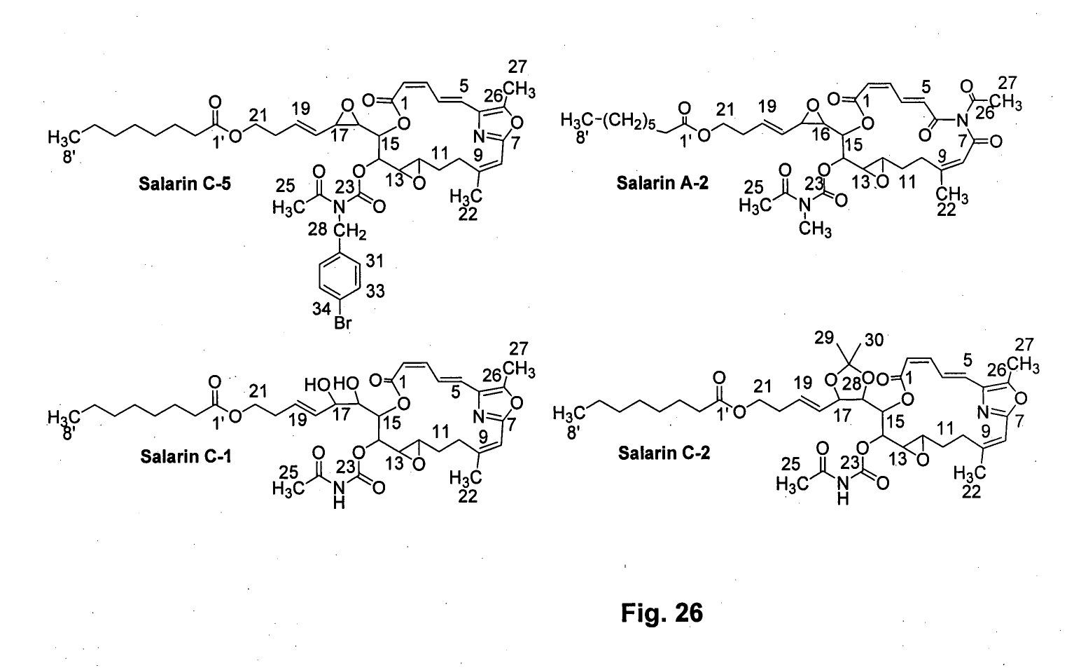

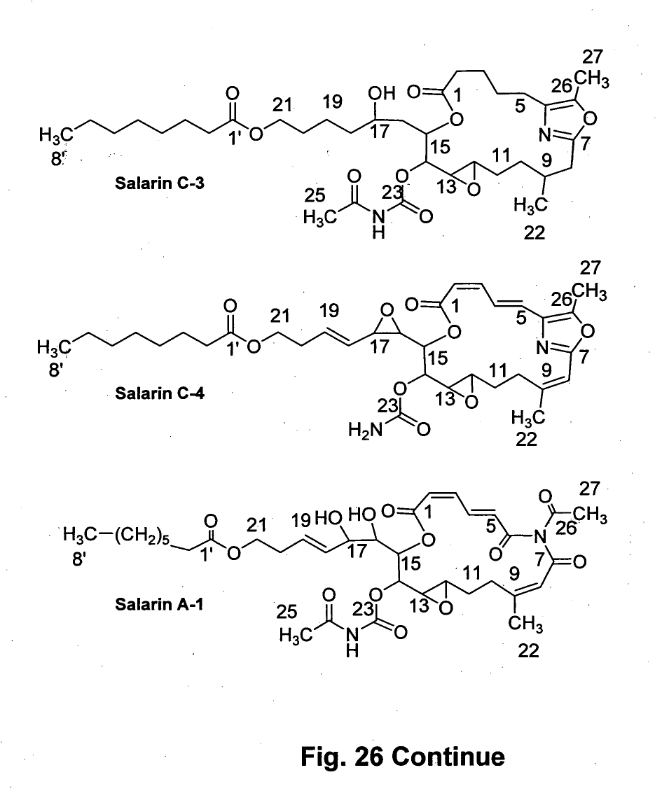

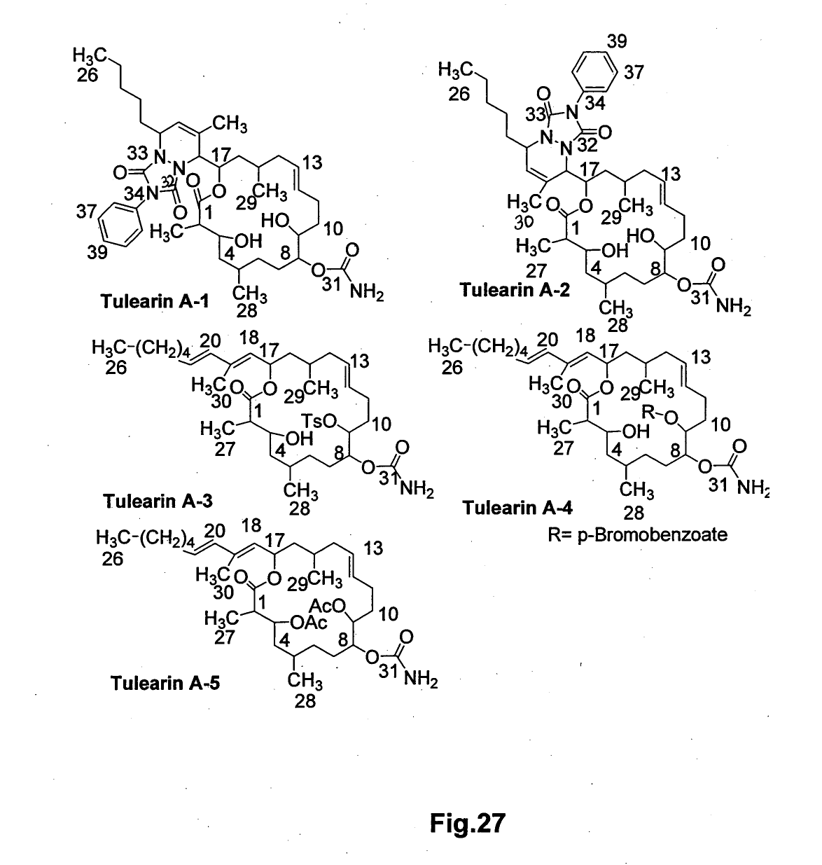

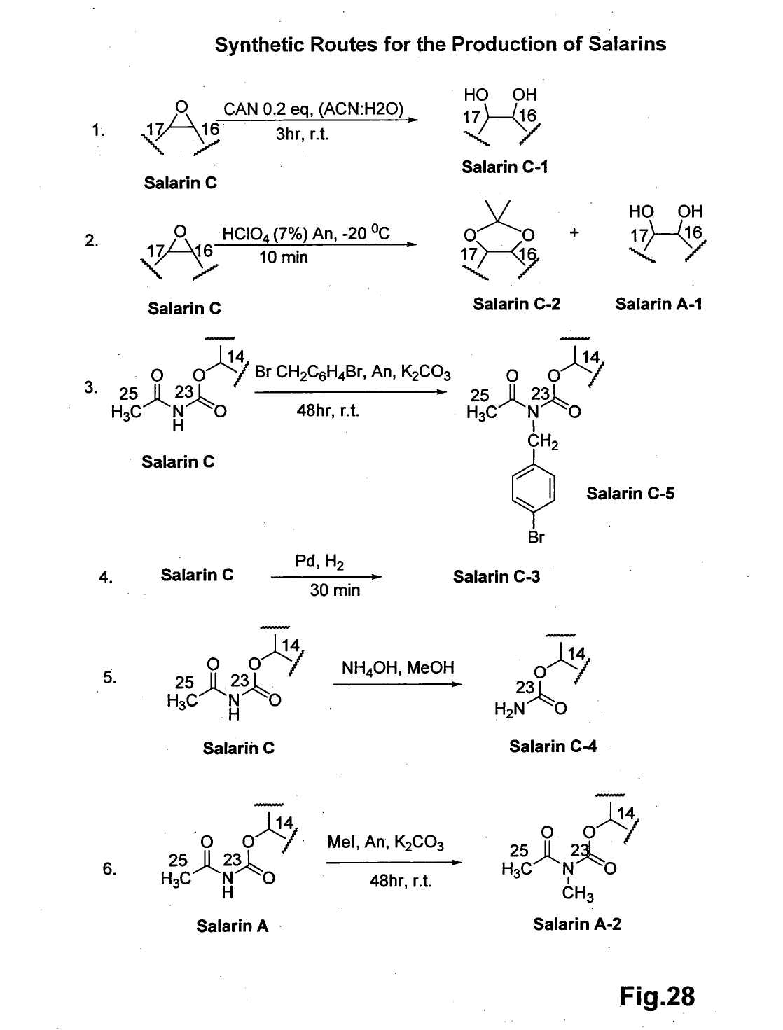

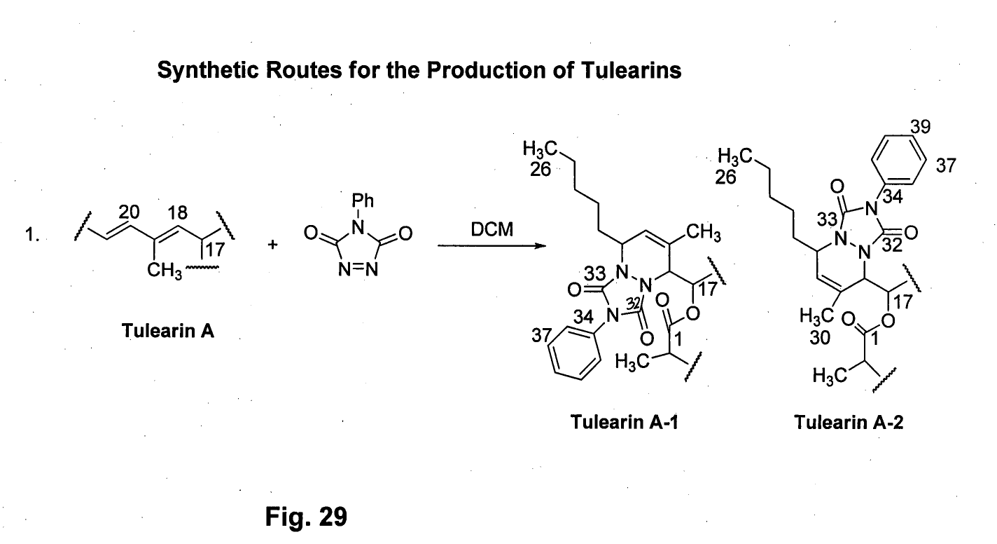

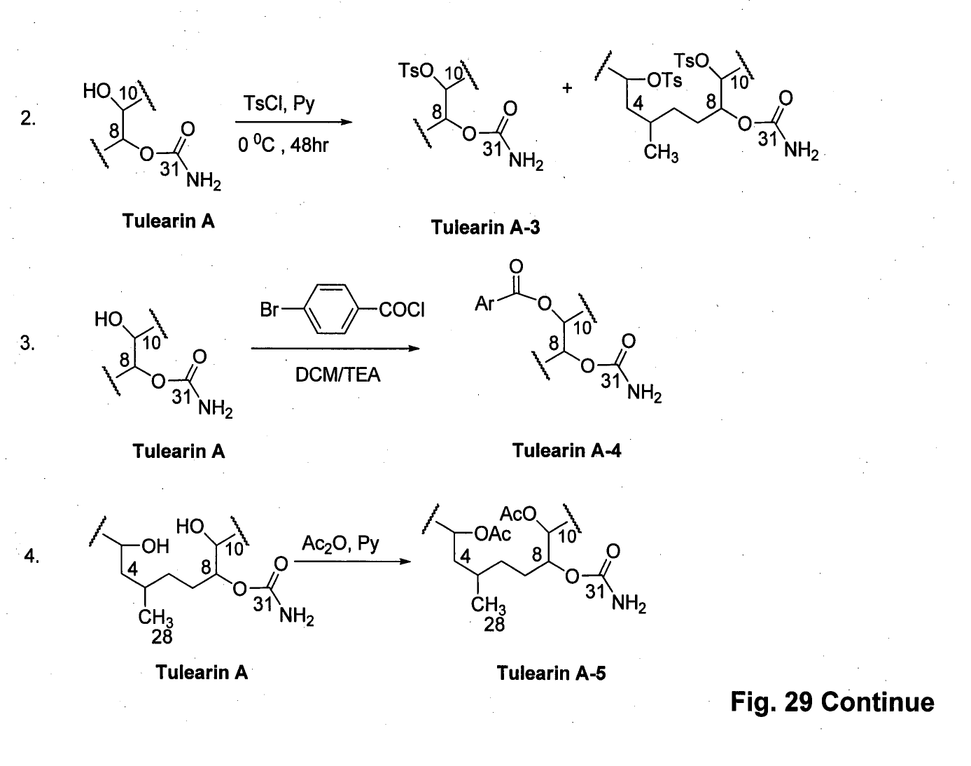

The potential effects of Salarin C on cell proliferation were also tested. Human leukemic cell lines UT-7 and K562, and the murine pro B cell line Ba/F3 which stably expresses the EPO receptor (EPO-R) were used as targets. FIG. 24A demonstrates that after 72 h in cull culture Salarin C at 0.5 μg/ml (1 μM) abolished nearly all viability of the three tested cell lines. In that respect, Salarin C was more potent than Salarins A and B and Tulearin A. FIG. 24B demonstrates that the anti-proliferative activity of Salarin C was dose-dependent. Salarin C at concentrations of 0.0005-0.5 μg/ml was added to the cells for 24 hours, and cell viability was determined by the MTT assay (FIG. 24B). ˜50% inhibition of cell proliferation was obtained at 0.5 μg/ml (1 μM) of Salarin C for the UT-7 cell line and at 0.05 μg/ml (0.1 μM) for the K562 cell line. Sensitivity of the Ba/F3 cells was much higher as their proliferation was completely arrested at 0.1 μM Salarin C (FIG. 24B). The effective concentration range of Salarin C (IC₅₀ 0.1-1 μM) is comparable, and even lower than that of other chemotherapeutic agents, e.g. geldanamycin (Jeon et al. (2007), J. Pathol., 213, 170-179). The effect of Salarin C on cell viability was also observed by microscopy (FIG. 25). Whereas the control cells had a normal morphology, cells treated over-night with Salarin C (0.01 μM or 0.2 μM) displayed lower viability and characteristics of apoptotic cells (cell shrinkage, nuclear condensation and fragmentation, and formation of apoptotic bodies).

1. A compound of the formula I, including salts, stereoisomers, geometrical isomers, solvates, and pharmaceutically acceptable salts thereof: 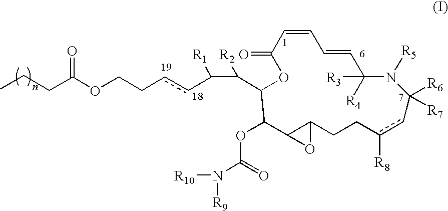
wherein:
R₁ and R₂ each independently is selected from null, —H, —OR₁₁, and —NR₁₂R₁₃, or
R₁ and R₂ together with the carbon atoms to which they are bonded form a heterocyclic ring system having 3 or 5 atoms, said heterocyclic ring comprising at least one heteroatom selected from O and N;
R₃ and R₄ each independently is selected from null, —H, —OR₁₄, and —C(O)C₁-C₆-alkyl;
or
R₃ and R₄ together with the carbon atom to which they are bonded form a group selected from a carbonyl group and C₆═CR₁₅R₁₆;
R₅ is selected from null, —H and —C(O)C₁-C₆-alkyl;
R₆ and R₇ each independently is selected from null, or together with the carbon to which they are bonded form a carbonyl group;
where R₃ and R₄ together form C₆═CR₁₅R₁₆ and where R₅ and one of R₆ and R₇ are absent, the N atom adjacent to C₇ forms a double bond with C₇ and the other of R₆ and R₇ and one of R₁₅ and R₁₆, together with the carbon atoms to which they are bonded, form a 5-membered heterocyclic ring, said heterocyclic ring comprising one or more atoms selected from N and O;
R₈ is selected from —H and C₁-C₆-alkyl;
R₉ is selected from —H, C₁-C₆-alkyl, C₁-C₆-alkylene-C₆-C₁₀-aryl and C₆-C₁₀-arylene-C₁-C₆-alkyl;
R₁₀ is selected from —H, C₁-C₆-alkyl and —C(O)R₁₇;
R₁₁ is selected from —H and C₁-C₆-alkyl;
R₁₂ and R₁₃ independently of each other is selected from —H and C₁-C₆-alkyl;
R₁₄ is selected from —H and C₁-C₆-alkyl;
R₁₅ and R₁₆ each independently is selected from —H and C₁-C₆-alkyl;
R₁₇ is a C₁-C₆-alkyl;
n is an integer from 0 to 12;
the C₈-C₉ bond is a single or double bond;
and
wherein the C₁₈-C₁₉ bond is a single or double bond.

2. The compound according to claim 1, wherein R₆ and R₇ together with the carbon to which they are bonded form a carbonyl group.

3. The compound according to claim 2, wherein R₁ and R₂ together with the carbon atoms to which they are bonded form a 3- or 5-membered heterocyclic ring system comprising at least one heteroatom selected from O and N.

4. The compound according to claim 3, wherein said heterocyclic ring system is selected from: 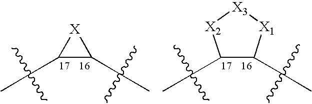
wherein
X is selected from —O—, —NH, and —N—C₁-C₆-alkyl;
X₁ and X₂ each independently is selected from —O—, —NH, —N—C₁-C₆-alkyl, CH₂, CHhal, C (hal)₂, CH(C₁-C₆-alkyl), C(C₁-C₆-alkyl)₂, CH(C₆-C₁₀-aryl) and C(C₆-C₁₀-aryl)₂; and
X₃ is selected from CH₂, CHhal, C(hal)₂, CH(C₁-C₆-alkyl), C(C₁-C₆-alkyl)₂, CH(C₆-C₁₀-aryl) and C(C₆-C₁₀-aryl)₂.

5. The compound according to claim 4, wherein said X and one of X₁ and X₂ are —O—.

6. The compound according to claim 1, being of the general Formula I-A: 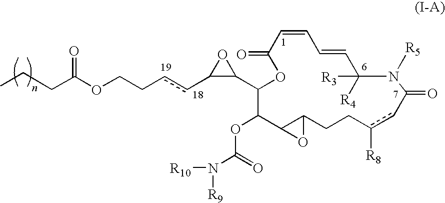
wherein R₃, R₄, R₅, R₈, R₉, R₁₀, and n are as defined in claim 1.

7. (canceled)

8. (canceled)

9. The compound according to claim 1, being of the general Formula I-B: 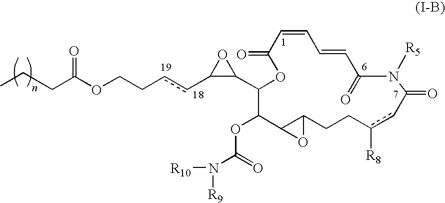
wherein R₅, R₈, R₉, R₁₀ and n are as defined in claim 1.

10-23. (canceled)

24. The compound according to claim 1 being of the general Formula I-C: 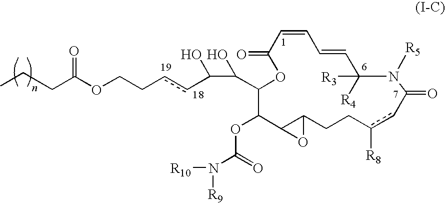
wherein R₃, R₄, R₅, R₈, R₉, R₁₀ and n are as defined in claim 1.

25-31. (canceled)

32. The compound according to claim 1, wherein:
R₁ and R₂ each independently is selected from null, —H, —OR₁₁, and —NR₁₂R₁₃, or
R₁ and R₂ together with the carbon atoms to which they are bonded form a 3- or 5-membered heterocyclic ring system comprising at least one heteroatom selected from O and N;
R₃ and R₄ together with the carbon atom to which they are bonded form the group C₆═CR₁₅R₁₆; R₁₅ and R₇ together with the carbon atoms to which they are bonded, form a 5-membered heterocyclic ring;
R₅ and R₆ are absent;
R₈ is selected from —H and C₁-C₆-alkyl;
R₉ is selected from —H, C₁-C₆-alkyl, C₁-C₆-alkylene-C₆-C₁₀-aryl and C₆-C₁₀-arylene-C₁-C₆-alkyl;
R₁₀ is selected from —H, C₁-C₆-alkyl and —C(O)R₁₇;
R₁₁ is selected from —H and C₁-C₆-alkyl;
R₁₂ and R₁₃ independently of each other is selected from —H and C₁-C₆-alkyl;
R₁₅ and R₁₆ each independently is selected from —H and C₁-C₆-alkyl;
R₁₇ is a C₁-C₆-alkyl;
n is an integer from 0 to 12;
the C₈-C₉ bond is a single or a double bond;
and
wherein the C₁₈-C₁₉ bond is a single or a double bond.

33. (canceled)

34. The compound according to claim 32, being of the general Formula I-D: 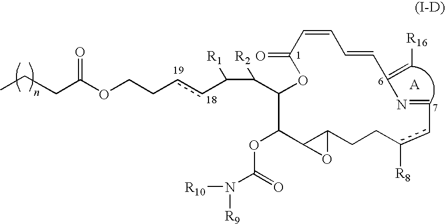
wherein R₁, R₂, R₈, R₉, R₁₀, R₁₆ and n are as defined in claim 9 and wherein ring A is a 5-membered ring having one additional heteroatom selected from N and O.

35. The compound according to claim 34, wherein said additional heteroatom is O.

36. The compound according to claim 34, wherein:
R₁ and R₂ together with the carbon atoms to which they are bonded form a 3- or 5-membered heterocyclic ring system comprising at least one heteroatom selected from O and N;
R₉ is selected from —H, C₁-C₆-alkyl, C₁-C₆-alkylene-C₆-C₁₀-aryl and C₆-C₁₀-arylene-C₁-C₆-alkyl;
R₁₀ is selected from —H, C₁-C₆-alkyl and —C(O)R₁₇;
R₁₅ and R₁₆ each independently is selected from —H and C₁-C₆-alkyl; and
R₁₇ is a C₁-C₆-alkyl.

37. The compound according to claim 34, wherein R₁ and R₂ together with the carbon atoms to which they are bonded form an epoxide ring.

38. The compound according to claim 34, being of the general formula I-E: 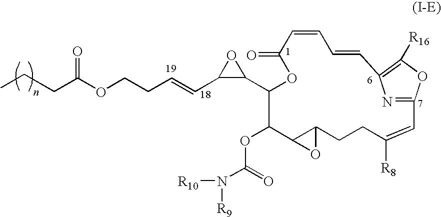
wherein R₆, R₉, R₁₀, R₁₆ and n are as defined in claim 34.

39. (canceled)

40. The compound according to claim 38, being of the general Formula I-F: 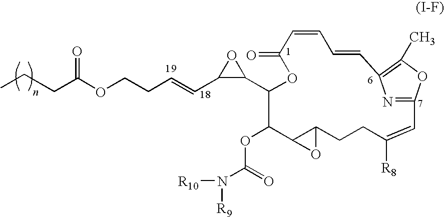
wherein R₈, R₁₀ and n are as defined in claim 38.

41-54. (canceled)

55. The compound according to claim 32, wherein said heterocyclic ring system is a 5-membered ring of the formula: 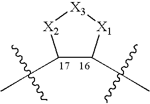
wherein at least one of X₁, X₂ and X₃ is —O— and the others of X₁, X₂ and X₃ are as defined in claim 4.

56-60. (canceled)

61. A compound of the general Formula II, including salts, stereoisomers, geometrical isomers, solvates, and pharmaceutically acceptable salts thereof: 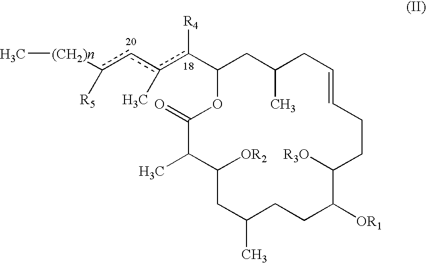
wherein;
R₁ is selected from —H, C₁-C₆-alkyl and —C(O)NR₆R₇;
R₂ and R₃ each independently is selected from —H, -Ts, C(O)NR₈R₉, —C(O) —C₁-C₆-alkyl and —C(O)—C₆-C₁₀-aryl;
R₄ is selected from —H, —O—, —OR₁₁, —N—, and NR₁₂R₁₃; and R₅ is selected from —H, —O—, —OR₁₄, —N—, and NR₁₅R₁₆
or
R₄ and R₅ together with the carbon atoms to which they are bonded form a heterocyclic ring system comprising at least one heteroatom selected from N and O; said ring system being a monocyclic or a multicyclic system; wherein each of said R₁₁, R₁₂, R₁₃, R₁₄, R₁₅ and R₁₆ is optionally a bond or an atom of said ring system or independently selected from —H and C₁-C₆ alkyl;
R₆, R₇, R₈ and R₉ each independently is selected from —H and C₁-C₆ alkyl;
n is an integer from 0-6;
the C₁₈-C₁₉ bond is a single bond or a double bond;
the C₁₉-C₂₀ bond is a single bond or a double bond; and
the C₂₀-C₂₁ bond is a single bond or a double bond.

62. The compound according to claim 61, wherein:
R₁ is selected from —H, C₁-C₆-alkyl and —C(O)NR₆R₇;
R₂ is selected from —H, -Ts, —C(O)—C₁-C₆ alkyl;
R₃ is selected from —H, -Ts, —C(O)NR₉R₁₀ and —C(O)—C₁-C₆ alkyl;
R₄ and R₅ are each —H;
R₆, R₇, R₈ and R₉ each independently is selected from —H and C₁-C₆-alkyl;
n is an integer from 0-6;
the C₁₈-C₁₉ bond is a single bond or a double bond;
the C₁₉-C₂₀ bond is a single bond; and
wherein the C₂₀-C₂₁ bond is a single or a double bond.

63. (canceled)

64. The compound according to claim 61 being of the general Formula II-A: 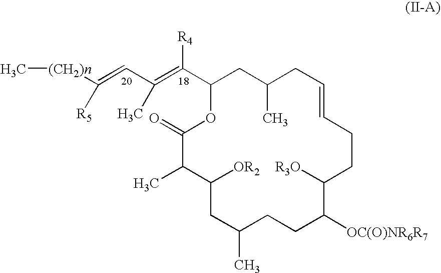
wherein R₂, R₃, R₄, R₅, R₇ and R₈ are as defined in claim 61.

65-70. (canceled)

71. The compound according to claim 61, being of the general Formula II-B: 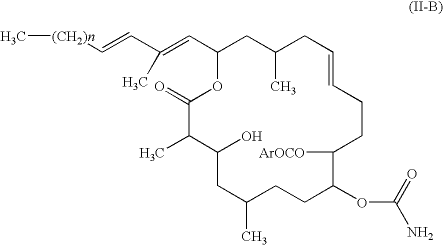
wherein Ar is a C₆-C₁₀-aryl, being optionally substituted substituted by at least one halide.

72-75. (canceled)

76. The compound according to claim 61, wherein
R₁ is selected from —H, C₁-C₆-alkyl and —C(O)NR₈R₇;
R₂ is selected from —H, -Ts, —C(O)—C₁-C₆ alkyl;
R₃ is selected from —H, -Ts, —C(O)NR₉R₁₀ and —C(O)—C₁-C₆ alkyl;
R₄ and R₅ together with the carbon atoms to which they are bonded form a heterocyclic ring system comprising at least one heteroatom selected from N and O; said ring system being a monocyclic or a multicyclic system;
R₆, R₇, R₈ and R₉ each independently is selected from —H and C₁-C₆-alkyl;
n is 0 or an integer from 1-6;
the C₁₈-C₁₉ bond and the C₂₀-C₂₁ bond are each a single bond;
and
the bond C₁₉-C₂₀ is a double bond.

77-82. (canceled)

83. A compound selected from the group consisting of:

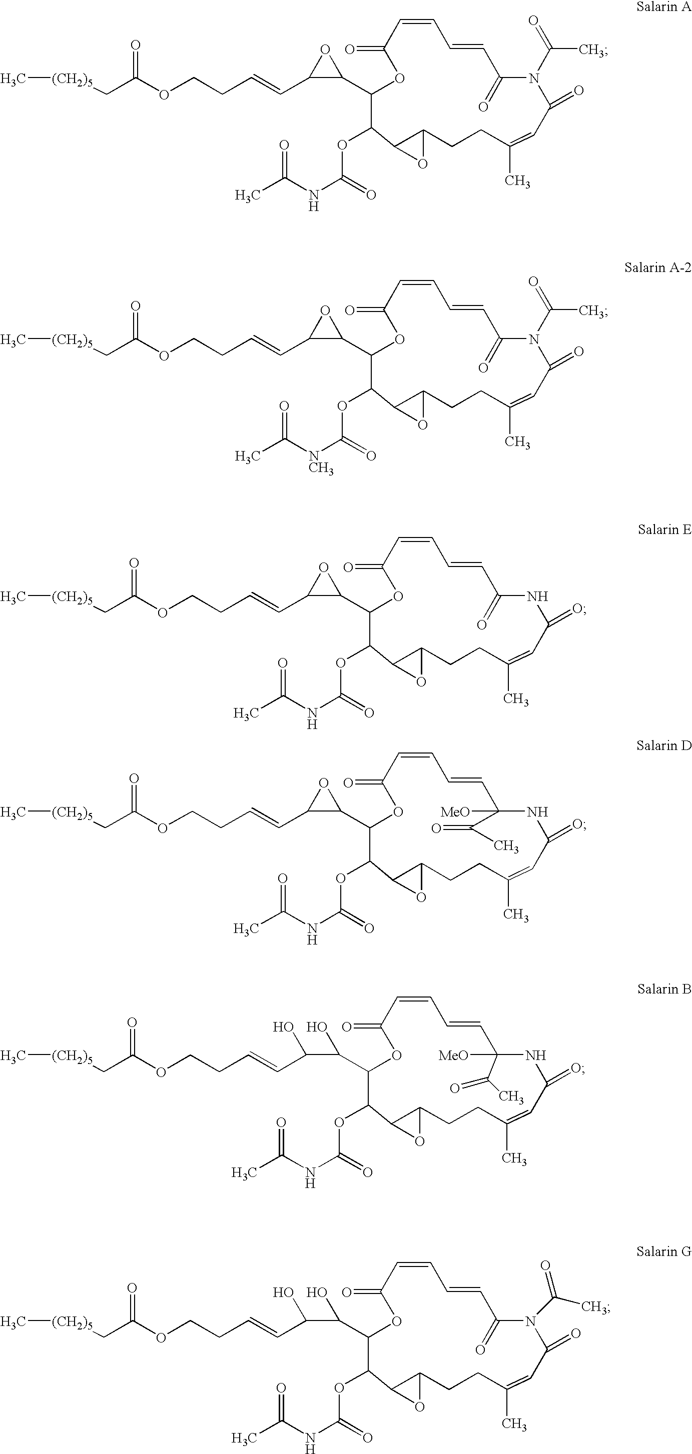

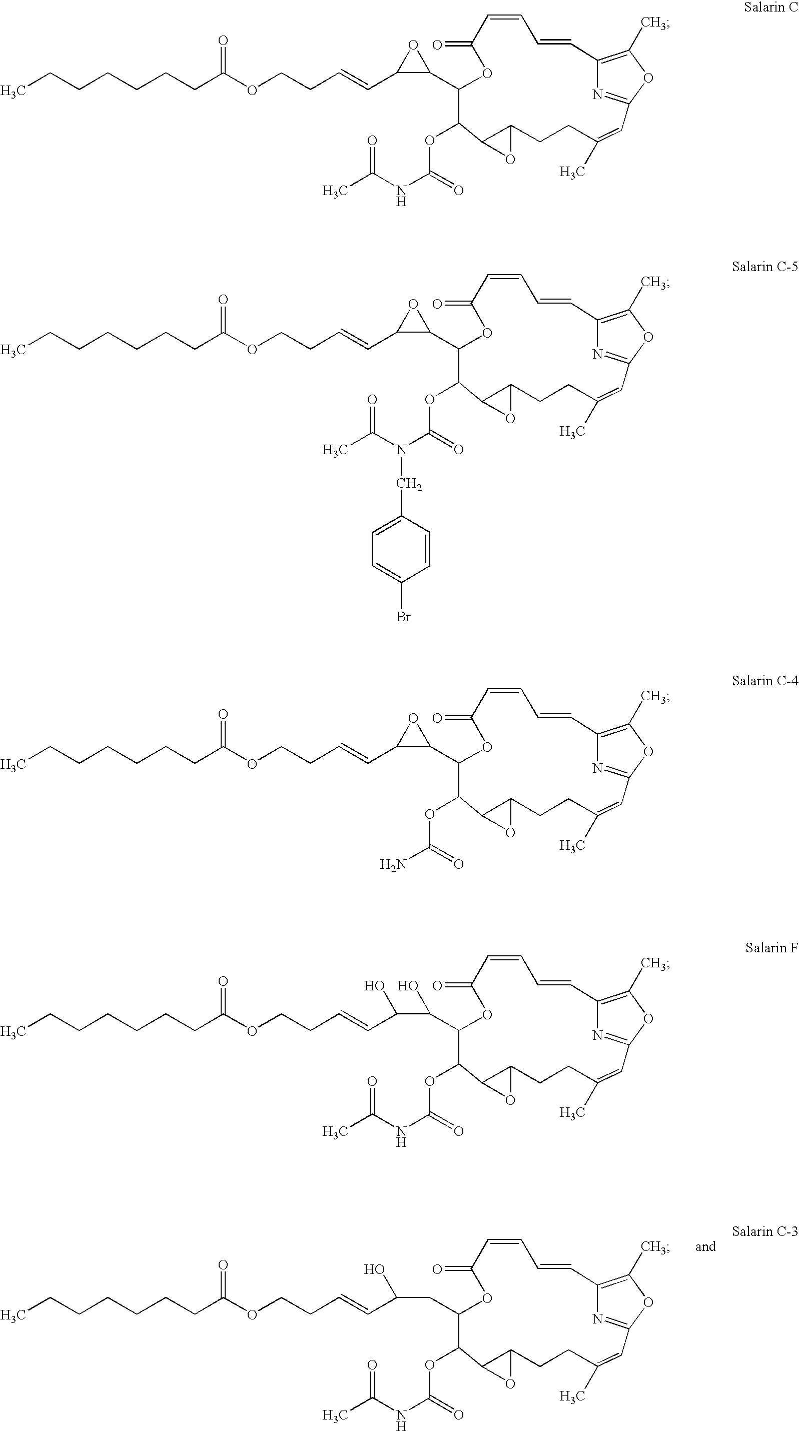

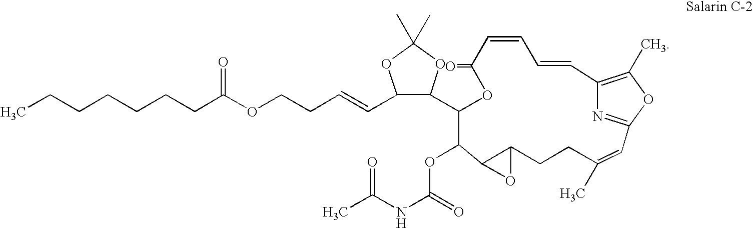

84-93. (canceled)

94. A composition comprising at least one compound of the general Formula I; 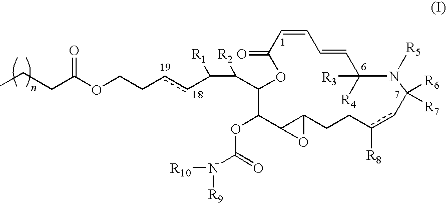
wherein:
R₁ and R₂ each independently is selected from null, —H, —OR₁₁, and —NR₁₂R₁₃, or
R₁ and R₂ together with the carbon atoms to which they are bonded form a heterocyclic ring system having 3 or 5 atoms, said heterocyclic ring comprising at least one heteroatom selected from O and N;
R₃ and R₄ each independently is selected from null, —H, —OR₁₄, and —C(O)C₁-C₆-alkyl;
or
R₃ and R₄ together with the carbon atom to which they are bonded form a group selected from a carbonyl group and C₆═CR₁₅R₁₆;
R₅ is selected from null, —H and —C(O)C₁-C₆-alkyl;
R₆ and R₇ each independently is selected from null, or together with the carbon to which they are bonded form a carbonyl group;
where R₃ and R₄ together form C₆═CR₁₅R₁₆ and where R₅ and one of R₆ and R₇ are absent, the N atom adjacent to C₇ forms a double bond with C₇ and the other of R₆ and R₇ and one of R₁₅ and R₁₆, together with the carbon atoms to which they are bonded, form a 5-membered heterocyclic ring, said heterocyclic ring comprising one or more atoms selected from N and O;
R₈ is selected from —H and C₁-C₆-alkyl;
R₉ is selected from —H, C₁-C₆-alkyl, C₁-C₆-alkylene-C₆-C₁₀-aryl and C₆-C₁₀-arylene-C₁-C₆-alkyl;
R₁₀ is selected from —H, C₁-C₆-alkyl and —C(O)R₁₇;
R₁₁ is selected from —H and C₁-C₆-alkyl;
R₁₂ and R₁₃ independently of each other is selected from —H and C₁-C₆-alkyl;
R₁₄ is selected from —H and C₁-C₆-alkyl;
R₁₅ and R₁₆ each independently is selected from —H and C₁-C₆-alkyl;
R₁₇ is a C₁-C₆-alkyl;
n is an integer from 0 to 12;
the C₈-C₉ bond is a single or double bond;
and
wherein the C₁₈-C₁₉ bond is a single or double bond.

95. The composition according to claim 94, being a pharmaceutical composition.

96-98. (canceled)

99. The composition according to claim 94, further comprising a pharmaceutically acceptable carrier.

100. A method for the treatment or prevention of a disease or disorder, said method comprising administering to a subject in need thereof a composition comprising a compound of the general Formula I 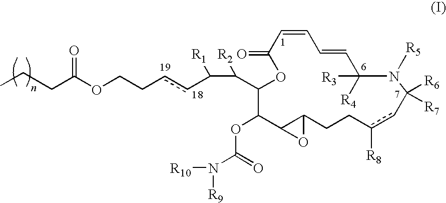
wherein:
R₁ and R₂ each independently is selected from null, —H, —OR₁₁, and —NR₁₂R₁₃, or
R₁ and R₂ together with the carbon atoms to which they are bonded form a heterocyclic ring system having 3 or 5 atoms, said heterocyclic ring comprising at least one heteroatom selected from O and N;
R₃ and R₄ each independently is selected from null, —H, —OR₁₄, and —C(O)C₁-C₆-alkyl;
or
R₃ and R₄ together with the carbon atom to which they are bonded form a group selected from a carbonyl group and C₆═CR₁₅R₁₆;
R₅ is selected from null, —H and —C(O)C₁-C₆-alkyl;
R₆ and R₇ each independently is selected from null, or together with the carbon to which they are bonded form a carbonyl group;
where R₃ and R₄ together form C₆═CR₁₅R₁₆ and where R₅ and one of R₆ and R₇ are absent, the N atom adjacent to C₇ forms a double bond with C₇ and the other of R₆ and R₇ and one of R₁₅ and R₁₆, together with the carbon atoms to which they are bonded, form a 5-membered heterocyclic ring, said heterocyclic ring comprising one or more atoms selected from N and O;
R₈ is selected from —H and C₁-C₆-alkyl;
R₉ is selected from —H, C₁-C₆-alkyl, C₁-C₆-alkylene-C₆-C₁₀-aryl and C₆-C₁₀-arylene-C₁-C₆-alkyl;
R₁₀ is selected from —H, C₁-C₆-alkyl and —C(O)R₁₇;
R₁₁ is selected from —H and C₁-C₆-alkyl;
R₁₂ and R₁₃ independently of each other is selected from —H and C₁-C₆-alkyl;
R₁₄ is selected from —H and C₁-C₆-alkyl;
R₁₅ and R₁₆ each independently is selected from —H and C₁-C₆-alkyl;
R₁₇ is a C₁-C₆-alkyl;
n is an integer from 0 to 12;
the C₈-C₉ bond is a single or double bond;
and
wherein the C₁₈-C₁₉ bond is a single or double bond;

101. The method according to claim 100, wherein said disease or disorder is associated with cell hyperproliferation.

102. The method according to claim 100, wherein said disease is cancer.

103. The method according to claim 100, wherein administration of said compound is in combination with at least one other medicament, chemotherapy, radiotherapy, or another treatment.

104. A compound selected from the group consisting of

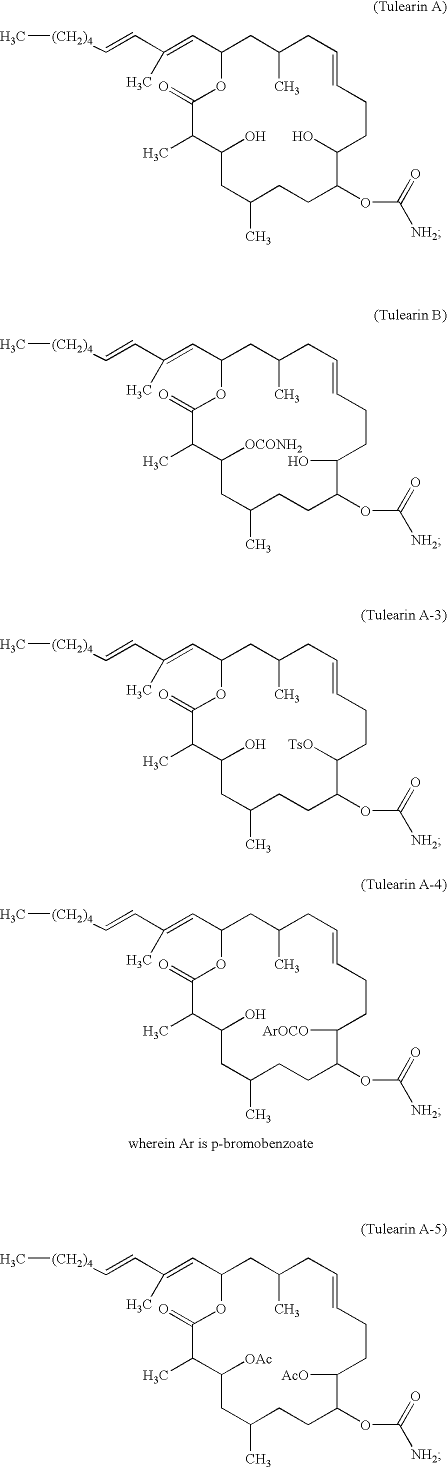

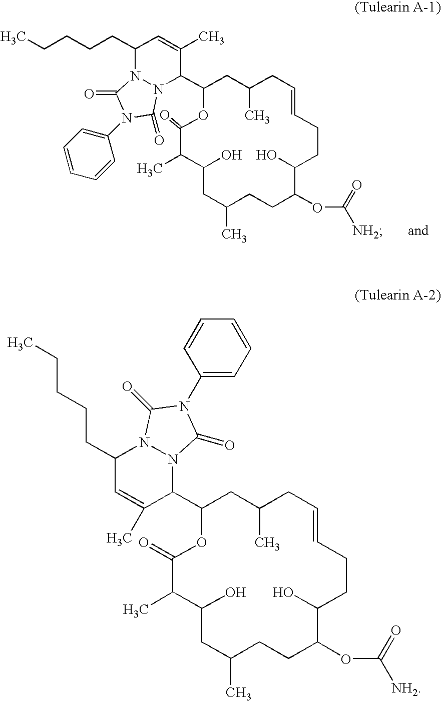

105. A composition comprising at least one compound of the general Formula II; 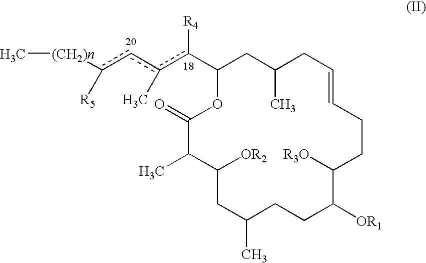
wherein;
R₁ is selected from —H, C₁-C₆-alkyl and —C(O)NR₆R₇;
R₂ and R₃ each independently is selected from —H, -Ts, —C(O)NR₈R₉, —C(O)—C₁-C₆-alkyl and —C(O)—C₆-C₁₀-aryl;
R₄ is selected from —H, —O—, —OR₁₁, —N—, and —NR₁₂R₁₃; and R₅ is selected from —H, —O—, —OR₁₄, —N—, and —NR₁₅R₁₆
or
R₄ and R₅ together with the carbon atoms to which they are bonded form a heterocyclic ring system comprising at least one heteroatom selected from N and O; said ring system being a monocyclic or a multicyclic system; wherein each of said R₁₁, R₁₂, R₁₃, R₁₄, R₁₅ and R₁₆ is optionally a bond or an atom of said ring system or independently selected from —H and C₁-C₆ alkyl;
R₆, R₇, R₈ and R₉ each independently is selected from —H and C₁-C₆ alkyl;
n is an integer from 0-6;
the C₁₈-C₁₉ bond is a single bond or a double bond;
the C₁₉-C₂₀ bond is a single bond or a double bond; and
the C₂₀-C₂₁ bond is a single bond or a double bond.

106. The composition according to claim 105, being a pharmaceutical composition.

107. The composition according to claim 105, further comprising a pharmaceutically acceptable carrier.

108. A method for the treatment or prevention of a disease or disorder, said method comprising administering to a subject in need thereof a composition comprising a compound of the general Formula II 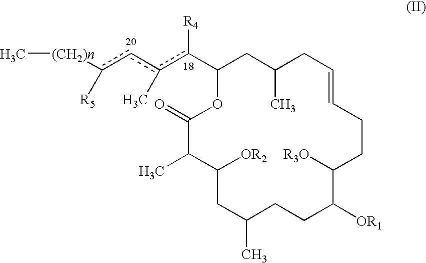
wherein;
R₁ is selected from —H, C₁-C₆-alkyl and —C(O)NR₆R₇;
R₂ and R₃ each independently is selected from —H, -Ts, —C(O)NR₈R₉, —C(O) —C₁-C₆-alkyl and —C(O)—C₆-C₁₀-aryl;
R₄ is selected from —H, —O—, —OR₁₁, —N—, and —NR₁₂R₁₃; and R₅ is selected from —H, —O—, —OR₁₄, —N—, and —NR₁₅R₁₆
or
R₄ and R₅ together with the carbon atoms to which they are bonded form a heterocyclic ring system comprising at least one heteroatom selected from N and O; said ring system being a monocyclic or a multicyclic system; wherein each of said R₁₁, R₁₂, R₁₃, R₁₄, R₁₅ and R₁₆ is optionally a bond or an atom of said ring system or independently selected from —H and C₁-C₆ alkyl;
R₆, R₇, R₈ and R₉ each independently is selected from —H and C₁-C₆ alkyl;
n is an integer from 0-6;
the C₁₈-C₁₉ bond is a single bond or a double bond;
the C₁₉-C₂₀ bond is a single bond or a double bond; and
the C₂₀-C₂₁ bond is a single bond or a double bond.

109. The method according to claim 108, wherein said composition is orally administered.

110. The method according to claim 108, wherein said disease or disorder is associated with cell hyperproliferation.

111. The method according to claim 108, wherein said disease is cancer.

112. The method according to claim 108, wherein administration of said compound is in combination with at least one other medicament, chemotherapy, radiotherapy, or another treatment.

113. The method according to claim 108, wherein said composition is orally administered.
